(19) United States
(12) Patent Application Publication
(10) Pub. No.: US 2026/0232230 A1
(43) Pub. Date: Aug. 13, 2026

(54) SYSTEMS, METHODS AND DEVICES FOR BIOPHYSICAL MODELING AND RESPONSE PREDICTION

(71) Applicant: January, Inc., Menlo Park, CA (US)

(72) Inventors: Parin Bhadrik DALAL, Palo Alto, CA (US); Salar RAHILI, Menlo Park, CA (US); Solmaz Shariat TORBAGHAN, Burlingame, CA (US); Saransh Agarwal, Pittsburgh, PA (US); Mehrdad YAZDANI, Santa Clara, CA (US)

(21) Appl. No.: 19/633,486

(22) Filed: Mar. 30, 2026

Related U.S. Application Data
(63) Continuation-in-part of application No. 19/326,943, filed on Sep. 12, 2025.
Application No. 19/326,943 is a continuation of application No. 16/785,436, filed on Feb. 7, 2020, now Pat. No. 12,433,511.
Application No. 16/785,436 is a continuation of application No. PCT/US2019/063788, filed on Nov. 27, 2019.
(60) Provisional application No. 62/773,134, filed on Nov. 29, 2018.
(60) Provisional application No. 62/773,125, filed on Nov. 29, 2018.
(60) Provisional application No. 62/773,117, filed on Nov. 29, 2018.

(51) Int. Cl. A61B 5/145; A61B 5/00; A61B 5/024; G06F 40/205; G06N 3/045; G06N 3/08; G06N 3/088; G16H 10/60; G16H 20/60; G16H 40/67; G16H 50/20; G16H 50/50; G16H 50/70

(52) U.S. Cl. CPC A61B 5/14532; A61B 5/0022; A61B 5/0024; A61B 5/024; A61B 5/6801; A61B 5/7275; G06F 40/205; G06N 3/045; G06N 3/08; G06N 3/088; G16H 10/60; G16H 20/60; G16H 40/67; G16H 50/20; G16H 50/50; G16H 50/70

(57) ABSTRACT

Various systems and methods are disclosed. One or more of the methods disclosed uses machine learning algorithms to predict biophysical responses from biophysical data, such as heart rate monitor data, food logs, or glucose measurements. Biophysical responses may include behavioral responses. Additional systems and methods extract nutritional information from food items by parsing strings containing names of food items.

### CROSS-REFERENCE

This application is a continuation-in-part of U.S. application Ser. No. 19/326,943, filed Sep. 12, 2025, which is a continuation of U.S. application Ser. No. 16/785,436, filed Feb. 7, 2020 (now U.S. Pat. No. 12,433,511, issued October 7, 2025), which is a continuation of International Application No. PCT/US 2019/063788, filed Nov. 27, 2019, which claims the benefit of U.S. Provisional Application No. 62/773,134, filed Nov. 29, 2018, U.S. Provisional Application No. 62/773,125, filed Nov. 29, 2018, and U.S. Provisional Application No. 62/773,117, filed Nov. 29, 2018, each of which is incorporated by reference herein in its entirety.

### TECHNICAL FIELD

The present disclosure relates generally to the systems for monitoring and evaluating the health of a subject, and more particularly to monitoring health and providing suggestions for improving health, via a personal electronic device, or the like.

### SUMMARY

In an aspect, the present disclosure provides a personalized glucose monitoring device for a test subject, comprising: a heart rate monitor configured to obtain time-series heart rate data of the test subject over a period of time; a continuous glucose monitor configured to obtain time-series blood glucose levels of the test subject over the period of time; computer memory configured to store a set of training data comprising food source data for a set of training subjects and glucose levels responsive to the food source data; and one or more computer processors operatively coupled to the computer memory, wherein the one or more computer processors are individually or collectively programmed to: (i) train a starting glucose prediction model with the set of training data to predict blood glucose levels in response at least in part to food source data, the starting glucose regulation model comprising population-based insulin resistance parameters, wherein the starting glucose regulation model comprises an artificial neural network configured to analyze time-series input data, wherein the artificial neural network comprises (i) an input layer configured to receive a sequence of the time-series input data and (ii) and one or more hidden layers that are trained to maintain a state across time increments and capture dependencies in the sequence of the time-series input data; (ii) obtain personalized glycemic response data of the test subject, wherein obtaining the personalized glycemic response data comprises obtaining the time-series heart rate data of the test subject from the heart rate monitor, and obtaining the time-series blood glucose levels of the test subject from the continuous glucose monitor; (iii) update the starting glucose regulation model with the personalized glycemic response data of the test subject, wherein the updating comprises adjusting the population-based insulin resistance parameters, until convergence is achieved between a predicted glycemic response of the test subject and an actual glycemic response of the test subject, thereby producing a personalized glucose regulation model for the test subject; (iv) generate a predicted blood glucose level for the test subject, using the personalized glucose regulation model for the test subject; (v) automatically generate a message containing the predicted blood glucose level for the test subject whenever an updated predicted blood glucose level has been generated; and (vi) transmit the message to all users of the personalized glucose monitoring device over a computer network in real time, so that each user has real-time access to up-to-date predicted blood glucose information for the test subject.

In some embodiments, the starting glucose regulation model comprises a differential equation model or a set of coupled equations.

In some embodiments, the one or more computer processors are individually or collectively programmed to further apply the personalized glucose regulation model for the test subject to at least personal food source data of the test subject to generate the predicted blood glucose level for the test subject.

In some embodiments, the one or more computer processors are individually or collectively programmed to further generate the predicted blood glucose level for the test subject in real time.

In some embodiments, the artificial neural network comprises a recurrent neural network (RNN).

In some embodiments, the artificial neural network is trained with data of a pre-determined population.

In some embodiments, (iii) further comprises classifying the test subject into a demographic equivalent group based at least in part on characteristic data of the test subject.

In some embodiments, the differential equation model comprises a food source function, a glucose production function, or a glucose uptake function.

In some embodiments, the one or more computer processors are individually or collectively programmed to further train the food source function using training data comprising glycemic responses of a population to pre-determined foods or glycemic responses calculated from data for pre-determined foods.

In some embodiments, the RNN comprises a long short-term memory (LSTM) RNN.

In some embodiments, the LSTM RNN comprises a set of LSTM units, wherein each of the set of LSTM units comprises a cell, an input gate, an output gate, and a forget gate.

In some embodiments, the one or more computer processors are individually or collectively programmed to further generate a recommended action for the test subject, based at least in part on the predicted blood glucose level for the test subject.

In some embodiments, the recommended action comprises a medical recommendation, a diet recommendation, a physical activity recommendation, a sleep recommendation, a hydration recommendation, or a stress release recommendation.

In some embodiments, the one or more computer processors are individually or collectively programmed to further: (vii) determine whether the predicted blood glucose level for the test subject has a deviation outside of response limits; and (viii) responsive to the determining in (vii), generate a notification indicating whether the predicted blood glucose level for the test subject has the deviation outside of the response limits.

In some embodiments, the notification is sent to the test subject.

In some embodiments, the notification is sent to a party different from the test subject.

In another aspect, the present disclosure provides a method for personalized glucose monitoring of a subject, comprising: (a) using a heart rate monitor to obtain time-series heart rate data of the subject over a period of time; (b) using a continuous glucose monitor to obtain time-series blood glucose levels of the subject over the period of time; (c) obtain a personalized glucose regulation model for the subject, wherein the personalized glucose regulation model is trained at least in part by: (1) obtaining a starting glucose prediction model configured to predict blood glucose levels in response at least in part to food source data, the starting glucose regulation model comprising population-based insulin resistance parameters, wherein the starting glucose regulation model is configured to analyze time-series input data; (2) obtaining personalized glycemic response data of the subject, wherein obtaining the personalized glycemic response data comprises obtaining the time-series heart rate data of the subject from the heart rate monitor, and obtaining the time-series blood glucose levels of the subject from the continuous glucose monitor; and (3) updating the starting glucose regulation model with the personalized glycemic response data of the subject, wherein the updating comprises adjusting the population-based insulin resistance parameters, until convergence is achieved between a predicted glycemic response of the subject and an actual glycemic response of the subject; (d) generate a predicted blood glucose level for the subject in real time, using the personalized glucose regulation model for the subject; (e) automatically generate a message containing the predicted blood glucose level for the subject whenever an updated predicted blood glucose level has been generated; and (f) transmit the message to a user set of the personalized glucose monitoring device over a computer network in real time, so that each user of the user set has real-time access to up-to-date predicted blood glucose information for the subject.

In an aspect, a method is disclosed. The method comprises obtaining a blood glucose level of a subject. When the blood glucose level of the subject deviates from a target response, the method further comprises determining a food consumed by the subject that corresponds to the deviated blood glucose level, and generating at least one alternative action for the subject. The at least one alternative action generates a blood glucose response in the subject with a reduced deviation from the target response as compared to a second glucose response generated by the subject consuming the food in absence of the at least one alternative action. The method further comprises electronically displaying at least the at least one alternative action.

In some embodiments, electronically displaying the blood glucose level displaying a visualization of blood glucose levels of the subject over a duration of time; and displaying the at least one alternate action comprises projecting a predicted glucose level for the subject for the at least one alternative action on the visualization with the blood glucose levels of the subject over the duration of time.

In some embodiments, the at least one alternative action comprises consuming at least one alternative food that reduces an elevation in blood glucose levels in the subject upon consumption by the subject as compared to not consuming the at least one alternative food.

In some embodiments, the at least one alternative action comprises performing at least one physical activity that reduces an elevation in blood glucose levels in the subject as compared to not performing the at least one physical activity.

In some embodiments, the at least one physical activity is exercising or fasting.

In some embodiments, the exercising is walking or running.

In some embodiments, the method further comprises monitoring the blood glucose level of the subject at least in part by using a continuous glucose monitoring device.

In some embodiments, the method further includes determining the food consumed by the subject comprises accessing a food log of the subject.

In some embodiments, generating the at least one alternative action comprises accessing logs of the subject to determine at least one previous action performed by the subject that reduced an elevation in blood glucose levels; and displaying the at least one alternative action comprises displaying a previous blood glucose response of the subject corresponding to the at least one previous action.

In some embodiments, accessing the logs of the subject comprises communicating over a network with a server system via an application executed by an electronic device.

In some embodiments, displaying the at least one alternative action comprises: displaying a plurality of alternative actions to the subject, receiving from the subject an alternative action selected by the subject from among the plurality of alternative actions, and displaying a predicted blood glucose response for the subject corresponding to the selected alternative action.

In some embodiments, displaying the at least one alternative action comprises generating a display that ranks a plurality of alternative actions according to predicted blood glucose level response for the subject.

In some embodiments, generating the at least one alternative action comprises applying data for the subject to a recommender system resident on at least one server system.

In an aspect, a system is disclosed. The system comprise a portable electronic device having a display. The system also comprises at least one application executable by the portable electronic device and configured to, in response to a determination that a subject's blood glucose level deviates from a target response, present on the display a graph of the subject's blood glucose level, a food consumed by the subject that corresponds to the deviated blood glucose level, and at least one alternative action for the subject, wherein the at least one alternative action generates a blood glucose response in the subject with a reduced deviation from the target response as compared to a second glucose response generated by the subject consuming the food in absence of the at least one alternative action.

In some embodiments, the application is further configured to present on the display a predicted subject blood glucose level response to the at least one alternative action displayed with the graph of the blood glucose level.

In some embodiments, the system further includes at least one metabolism model for the subject configured the generate the predicted subject blood glucose response.

In some embodiments, the system further includes: a memory system configured to store blood glucose responses of the subject corresponding to prior actions of the subject; and the predicted subject blood glucose response is generated from a prior blood glucose response of the user.

In some embodiments, the system further includes at least one remote computing system in communication with the portable electronic device that includes a recommender system configured to generate the at least one alternative action in response to input subject data; and the input subject data includes previous actions of the subject.

In some embodiments, the at least one alternative action includes an alternative food selected to generate the lower rise in blood glucose level than the food consumed.

In some embodiments, the at least one alternative action includes a physical activity selected to generate the lower rise in blood glucose level.

In some embodiments, the system further includes: a continuous glucose monitor in communication with the portable electronic device and configured to provide blood glucose levels thereto.

In some embodiments, the at least one application is configured to present a plurality of alternative actions on the display for selection by the subject, and when an alternative action is selected by the subject, display a predicted blood glucose response for the subject corresponding to the alternative action.

In some embodiments, the at least one application is configured to display a plurality of alternative actions ranked according to predicted blood glucose level response for the subject

A non-transitory computer-readable medium storing instructions that, when executed by a computing device, cause the computing device to perform operations comprising: obtaining a blood glucose level of a subject; when the blood glucose level of the subject deviates from a target response, determining a food consumed by the subject that corresponds to the deviated blood glucose level, and generating at least one alternative action for the subject, wherein the at least one alternative action generates a blood glucose response in the subject with a reduced deviation from the target response as compared to a second glucose response generated by the subject consuming the food in absence of the at least one alternative action; and electronically displaying at least the at least one alternative action.

### BRIEF DESCRIPTION OF THE DRAWINGS

The novel and inventive features of the invention are set forth with particularity in the appended claims. A better understanding of the features and advantages of the present invention will be obtained by reference to the following detailed description that sets forth illustrative embodiments, in which the principles of the invention are utilized, and the accompanying drawings which in this provisional patent application are provided in the Examples section below.

FIGS. 1A and 1B are flow diagrams of methods for detecting and displaying blood glucose (BG) spiking events according to embodiments.

FIGS. 1C to 1E are application displays for displaying and analyzing BG level spiking events according to embodiments.

FIGS. 2A and 2B are flow diagrams of methods for generating food alternatives to reduce BG levels according to embodiments.

FIGS. 2C to 2E are application displays for presenting food alternatives to reduce BG levels according to embodiments.

FIG. 3A is a flow diagram of a method for tracking and displaying health related events according to embodiments.

FIG. 3B is an application display for showing a display for presenting health related events according to embodiments.

FIG. 4A is a flow diagram of a method for generating “insight” cards according to a health improvement program according to embodiments.

FIGS. 4B to 4E are application displays of insight cards according to embodiments.

FIG. 5A is a flow diagram of a method for determining and displaying time-in-range (TIR) blood glucose data according to an embodiment.

FIG. 5B is an application display for showing BG TIR data according to embodiments.

FIGS. 5C and 5D are flow diagrams of methods for recommending an activity in response to heath data according to embodiments.

FIG. 5E is an application display for showing the presentation of a recommended activity according to embodiments.

FIGS. 5F and 5G are flow diagrams of methods for generating meal alternatives in response to health data according to embodiments.

FIGS. 5H-0 to 5H-2 are application displays for generating meal alternatives according to embodiments.

FIGS. 6A and 6B are flow diagrams of methods for detecting omissions in a food log and filling such omissions according to embodiments.

FIGS. 6C to 6D-3 are application displays for filling food log omissions according to embodiments.

FIGS. 7A and 7B are flow diagrams of methods for recording and displaying fasting periods according to embodiments.

FIG. 7C is an application display for displaying fasting data according to embodiments.

FIGS. 8A and 8B are flow diagrams of methods for generating and comparing BG TIR data for periods of calorie restriction and no calorie restriction according to embodiments.

FIG. 8C is an application display for showing BG TIR data for periods of calorie restriction and no calorie restriction according to embodiments.

FIGS. 9A to 9E are block diagrams of systems and methods for generating a metabolic score and recommendations according to embodiments.

FIG. 9F is a flow diagram of a method for determining and displaying gut health according to embodiments.

FIG. 9G is a flow diagram of a method for determining a data coverage score for health data according to embodiments.

FIGS. 9H-0 to 9H-2 are application displays for presenting metabolic score data according to embodiments.

FIGS. 9I-0 to 9I-2 are application displays for presenting health data coverage values according to embodiments.

FIG. 10A is a block diagram of a system for generating a health lifestyle score according to embodiments.

FIG. 10B is a flow diagram of a method for generating and displaying a health lifestyle score according to embodiments.

FIG. 10C is an application displays for presenting a health lifestyle score according to embodiments.

FIG. 11A is a flow diagram of a method for generating and presenting a wide range of health information for display according to embodiments.

FIG. 11B is an application display for presenting a wide range of health information according to embodiments.

FIG. 11C is a block diagram of food analysis system according to an embodiment.

FIG. 11D to 11F are flow diagrams of methods for generating and displaying nutrition information according to embodiments.

FIG. 11G is an application display for presenting a calorie intake and burn information according to embodiments.

FIGS. 11H and 11I are application displays for presenting a water intake information according to embodiments.

FIGS. 11J and 11K are application displays for presenting fiber intake information according to embodiments.

FIG. 12 is a block diagram of a system according to an embodiment.

FIG. 13 is a block diagram of a subject device according to an embodiment.

FIG. 14 shows a computer system that is programmed or otherwise configured to implement methods provided herein.

### DETAILED DESCRIPTION

As used in this document, the singular forms “a,” “an,” and “the” include plural references unless the context clearly dictates otherwise. Unless defined otherwise, all technical and scientific terms used herein have the same meanings as commonly understood by one of ordinary skill in the art. As used in this document, the term “comprising” means “including, but not limited to.”

Embodiments can include methods and systems for monitoring and presenting data on various aspects of health, including but not limited to blood glucose levels, fasting periods, nutrition (e.g., calorie intake, water intake, fiber intake), and fasting duration. In some embodiments, systems include, or operate with blood glucose monitors, such as continuous blood glucose monitors (CGM), and activity monitors, such as heart rate (HR) monitors or other activity monitors. In addition, embodiments can generate or receive food intake data from a subject, including but not limited to food log data.

According to embodiments, in response to data from a subject, relatively simple and easy to understand values (i.e., scores) that represent an aggregation of data can be presented to a subject. Some such scores can enable a subject to evaluate their health on a broad level, while others can provide data on more narrow aspects of health. Scores can be specific to the subject.

According to embodiments, in response to data from a subject, suggestions can be generated that present alternate choices, selected to provide better health outcomes for the subject. In response to user actions/choices, counterfactual actions/choices can be presented that illustrate what health consequences could have resulted from the counterfactual case.

A subject may be a human subject, which may be a child, adult, or elderly person. A subject may be a nonhuman subject, such as a mammalian subject. The subject may be a non-mammalian subject (another type of animal).

### Glucose Level Spiking

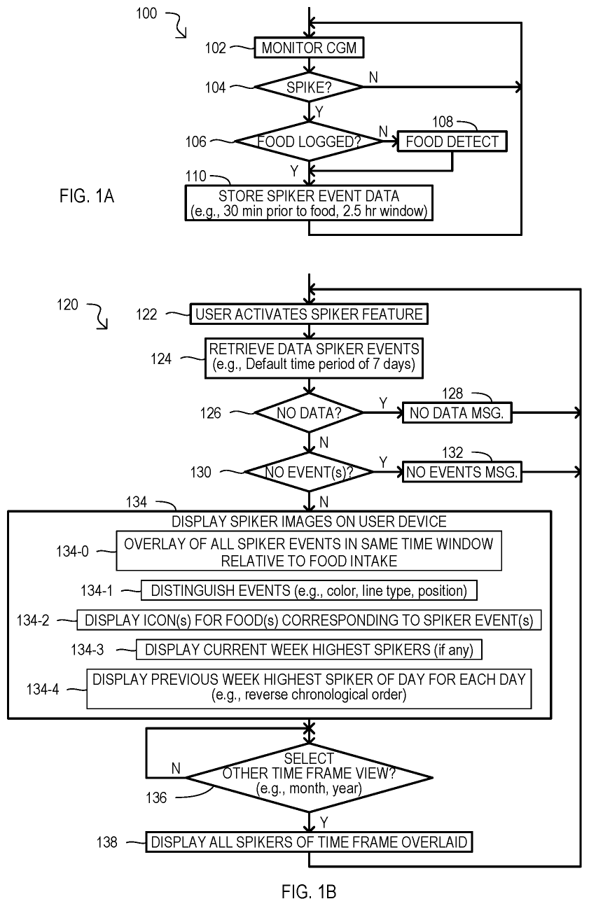

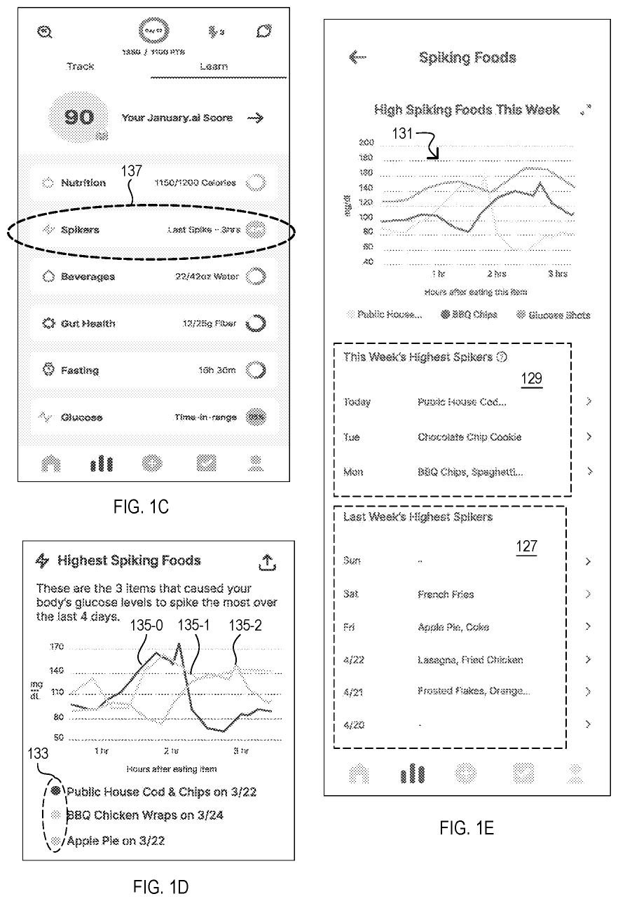

FIG. 1A shows a method 100 for detecting and recording increases in blood glucose levels that exceed a predetermined rate or magnitude (i.e., spikes). Spikes can be associated with a time period (e.g., data can be timestamped). A method 100 can include monitoring a glucose level of a subject 102. Such an action can include any suitable monitoring method, and in the embodiment shown, can include monitoring with a CGM. In some embodiments, blood glucose (BG) levels from a CGM can be automatically transmitted to remote location for storage (e.g., on a server).

A method 100 can determine if there has been a spike 104. Such an action can include comparing glucose levels to predetermined limits, including rate of change, magnitude or combinations thereof. That is, a spike 104 can be a deviation from a target response. In some embodiments, limits can be specific to a subject. In some embodiments, limits can be based, at least in part, on a clinical evaluation. Determining a spike has occurred can be performed on a server located remote from a subject, on a personal electronic device of a subject (e.g., smartphone), or a combination thereof. If no spike is detected (N from 104), a method 100 can continue to monitor glucose levels.

If a spike is detected (Y from 104), a method 100 can determine if a food has been logged corresponding to the spike 106. Such an action can include examining food log data in a predetermined time window prior to the spike. If no food has been logged (N from 106), a method 100 can execute a food detection procedure 108. A food detection procedure 108 can attempt to determine a food corresponding to the spike. Food detection methods according to embodiments will be described herein. However, any suitable food detection method can be used.

If food corresponding to the spike has been determined (Y from 106) or a food detection procedure has determined such a food, a method can store data for the spike event 110. In some embodiments, this can include storing glucose levels for a predetermined time window as well as the food corresponding to the spike.

The various determinations made in method 100 can be performed on a server located remote from a subject, on a personal electronic device of a subject (e.g., smartphone), or a combination thereof.

FIG. 1B shows a method 120 of presenting glucose level spike (i.e., spiker) events to a subject. A method 120 can include a subject activating a spiker feature of a device 122. Such an action can include a subject activating a feature of an application (app) on a personal electronic device. Data for spiker events can be retrieved 124. In some embodiments, such data can be for a predetermined amount of time (e.g., the current week and possibly a previous week). In some embodiments, such an action can include an app attempting to retrieve spiker data stored in a server. However, alternate embodiments can include retrieving all or a portion of the spiker data from the electronic device.

If no data can be found (Y from 126), a message can be generated indicating so 128. If data can be found (N from 126), but the data includes no spiker events (Y from 130), a message can be generated 132. In some embodiments, such a message can be an encouragement message, and/or include some other form of positive reinforcement.

If data for a spiker even can be found (N from 130), spiker data can be displayed for the subject as images 134. Such an action can include presenting spiker data in any suitable visual form on an electronic device, including a personal electronic device of the subject. In FIG. 1B, spiker images can include an overlay of all spiker events in a same time window relative to food intake 134-0. Such a display can provide a simple, easy to understand, comparative presentation of all spiker events. Different spiker events can be distinguished from one another 134-1. For example, spiker events can be presented as graphs of glucose level versus time, with different spiker events being distinguishable (e.g., different colors, line type, position). An icon can be presented representing the food(s) corresponding to the spiker event 134-2. In the embodiment shown, a current week's highest spiker event can be displayed or identified separately.

In some embodiments, displaying spiker images on a user device can also include displaying a current week's highest spikers (if any) 134-3 and/or displaying a previous weeks highest spiker of each day 134-4. In some embodiments, previous weeks highest spiker can be presented in reverse chronological order.

A method 120 can include enabling a subject to select a different time frame for viewing spiker events 136. Time frames can vary from a default time frame and can be longer and/or shorter than a default time frame. If a subject selects a different time frame (Y from 136), spiker data for the time frame can be displayed overlaid with one another 138. Such a display can take the form of any of those described herein or equivalent. If a subject does not select a different time frame (N from 136), a method 120 can return to 102.

FIG. 1C shows an interactive display (e.g., card) according to an embodiment. The interface can present various health related data for a user on a single display. In the embodiment shown, a “spikers” selection 137 can display the last recorded spiker event (3 hrs) before. A subject can activate the spiker selection to launch a spiker function as described herein or an equivalent. In some embodiments, an interface can be a screen of an app executed on a subject's electronic device.

FIG. 1D shows an interactive spiker display according to an embodiment. Such a spiker display can be generated by a method like that of FIG. 1B, or an equivalent. The display shows three spiker events in a graph 135-0/1/2 superimposed on one another. Each event is represented by a different colored response. Each spiker event can be presented from a common starting timepoint, such as when corresponding food was consumed. In addition, the display presents food corresponding to each spiker event with a circle 133 having a color matching the graphed event.

FIG. 1E shows another spiker display according to an embodiment. Such a spiker display can be generated by a method like that of FIG. 1B, or an equivalent. The display shows three highest spiking events that have occurred in a current week. Such data can be presented in a graph 131 like that of FIG. 1D, including identifying the foods corresponding to the spiker events 129. FIG. 1D also shows the events in text form and allows such events to be selected. Selecting an event can provide data for the event, including but not limited to, the food eaten, the day, the time, the location, and more detailed information on glucose levels. FIG. 1E also shows highest spiker events for the previous week 127 and allows such events to be selected.

### Food Alternative Recommender

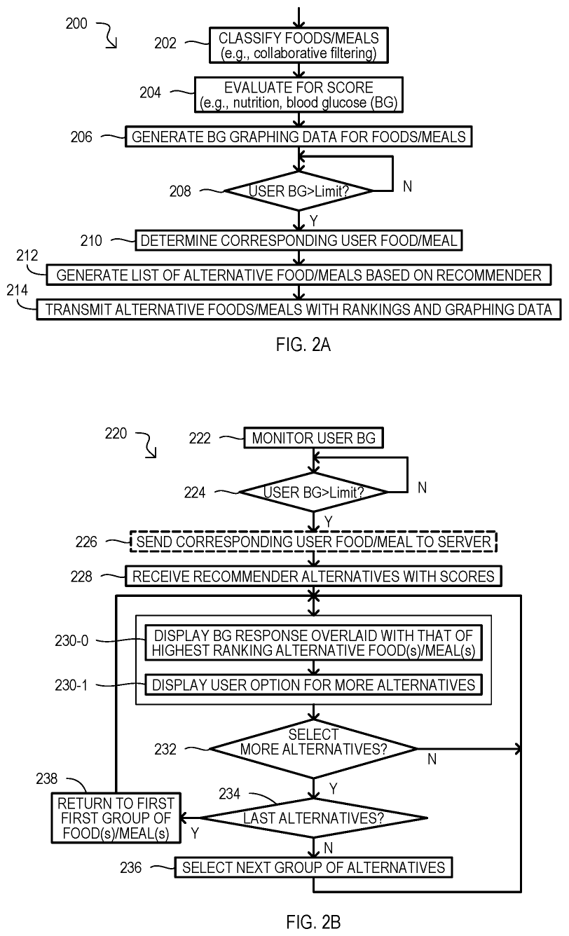

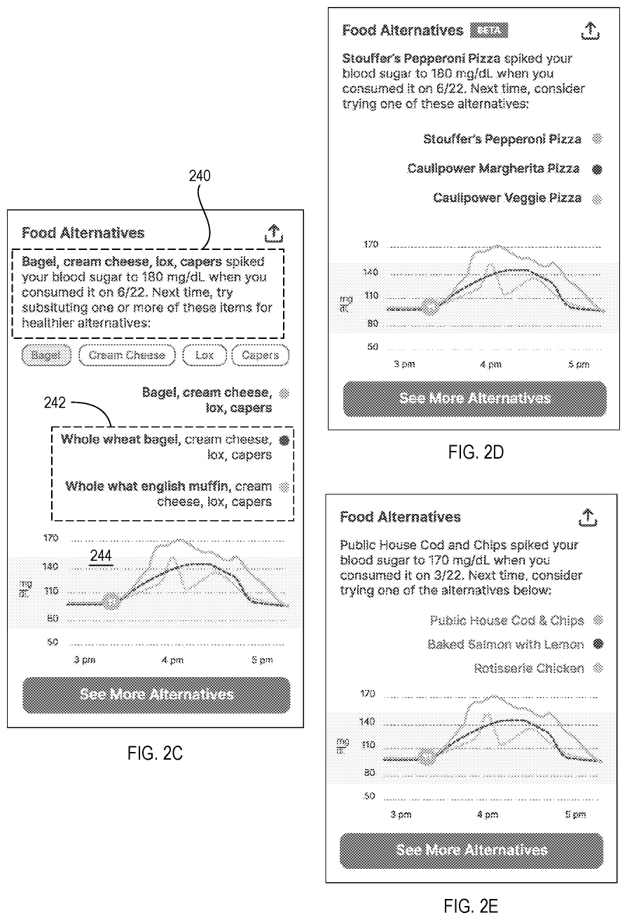

FIG. 2A shows a method 200 according to another embodiment. A method 200 can receive subject glucose levels, and if such levels exceed a limit, recommend an alternative food to that which was eaten. A method 200 can include classifying foods/meals (referred to as “foods”) 202. Such an action can include classifying foods according to taste. Such classification can include any suitable method, and in some embodiments can include collaborative filtering. However, such classification can also include a subject's preferences, either as provided by a subject, or as learned (via a statistical model) from a subject's food choices. Foods can be evaluated for a score 204. A score can correspond to a health effect of a food (glucose effect, such as glycemic index or load and/or nutrition). A method 200 can include generating graph data for foods 206. Such an action can include generating a glucose level response for a subject in response to the food. A response can be based on any suitable model, including but not limited to: models based on machine learning, models based on cohort responses, preexisting models based on a demographic match the subject, and/or previous responses of the subject to same or similar foods.

A method 200 can determine if a subject's glucose level exceeds a limit 208. Such an action can include receiving glucose from a subject, such as from a CGM or the like. A glucose level can take the form of any of those described herein, and can be particular to a subject. If a glucose level remains below a limit (N from 208), it can continue to be monitored.

If a glucose level exceeds a limit (Y from 208), food(s) corresponding to the glucose increase can be determined 210. Such an action can include accessing food log data for a subject and/or a food detection function as described herein or an equivalent. In some embodiments, such an action can correspond to detecting a spiker event. A recommender can generate a list of alternative food choices 212. Alternative food choices can be foods determined to be similar to that determined in 200 (i.e., recommended alternative foods to that which created the increase in glucose). A recommender take any suitable form, such as collaborative filtering, however recommended foods will have a better health effect (i.e., better score as determined in 204). The alternative foods can be transmitted to a subject with a ranking and graphing data 214. A Ranking can be based on the score, noted above. Graphics data can include an expected glucose response for the recommended foods.

FIG. 2B is a flow diagram of another method 220 according to another embodiment. A method 220 can be executed by devices of a subject to elicit recommended alternatives (e.g., food/meals), such as those generated by a method 200 of FIG. 2A, or an equivalent.

A method 220 can monitor a subject (i.e., user) glucose (e.g., BG) level 222. Such an action can include any of those described herein, or equivalents, including by way of a CGM. A method 220 can determine if a user glucose level exceeds a limit 224. Such an action can include those described herein or equivalents. If a glucose level remains below a limit (N from 224), the glucose level can continue to be monitored.

If a glucose level exceeds a limit (Y from 224), alternative foods generated by a recommender can be received 228. Such an action can include any of those described for 212 of FIG. 2A or equivalents. Optionally, in response to glucose exceeding a limit, a method 220 can transmit food data corresponding to the elevated glucose level 226. Upon receiving recommended alternatives, a glucose response corresponding to the elevated level can be displayed, overlaid with the glucose responses of the highest recommended alternative food 230-0. Such an action can distinguish between the different glucose responses as described herein (e.g., different colors). A user option for selecting alternative foods/meals can be displayed 230-1.

In addition, a subject can be given the option to select additional alternatives 232. If a subject does not choose alternatives (N from 232), the current alternative can continue to be displayed. If the subject does choose more alternatives (Y from 232), if a last alternative is reached (Y from 234), a method 220 can return to the first alternative 238 and display corresponding data. If a last alternative has not been reached (N from 234), the next set of alternatives can be selected 236, and overlay data for the selected alternative can be displayed (return to 230-0).

FIG. 2C shows an interactive display according to an embodiment. A display can be presented on a subject device in a method like that of FIG. 2B. The display shows, generally from top to bottom, a food eaten 240 that has led to an undesirably high increase in blood sugar. Ingredients of the food/meal are displayed and can be selected for more information. Alternatives 242, which can provide a better blood glucose response are listed. Such alternatives can be generated by a recommender as described herein. A display can include a graph 244 showing a subject's blood glucose corresponding to the eaten food overlaid with projected blood glucose levels for one or more recommended alternatives.

FIGS. 2D and 2E are additional examples of interactive displays like that shown in FIG. 2C, but for different foods.

### Event Tracker

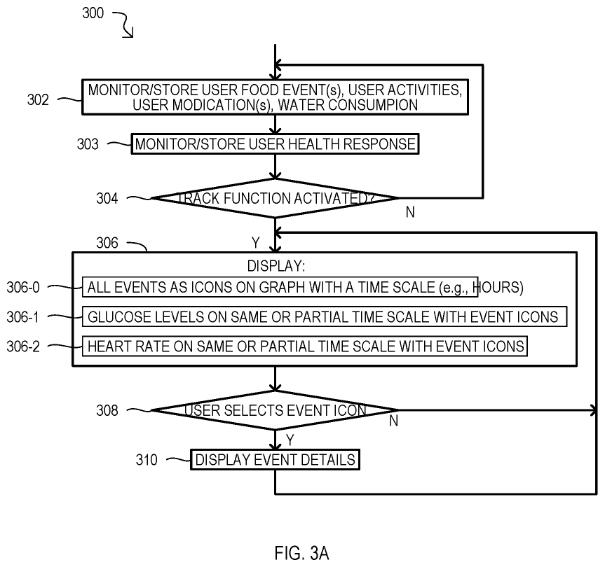

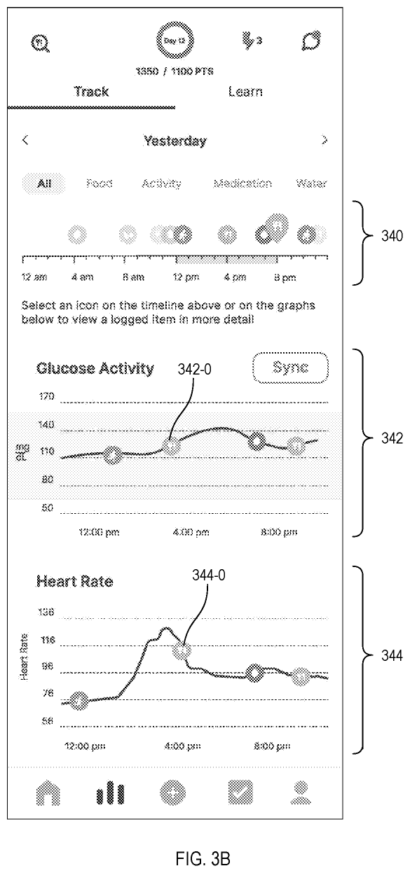

FIG. 3A is a flow diagram of a method 300 according to a further embodiment. A method 300 can monitor and store a subject's health related activities and provide a graphical representation of the activities in relation to health responses. Different activities can be represented as icons on a graphic representation of health responses. A method 300 can include monitoring various activities of a subject 302. Monitored activities can include, but are not limited to: food events, physical activities, doses of medication, and water/beverage consumption. Such an action can include any of those described herein, or equivalents, including but not limited to: logs with data for a subject (e.g., food logs, activity logs, medication logs) with the data being entered manually and/or automatically. A method 300 can also monitor and store health responses of a subject 303. Such an action can include monitoring any suitable health response, and in some embodiments can include monitoring a blood glucose level and HR according to any of the embodiments described herein. If a track function is not activated (N from 304), a subject's activities and health response can continue to be monitored and stored.

If a track function is activated (Y from 304), a display can be generated on a subject device 306. A track function can be activated in any suitable fashion, including a subject selecting a feature of an app running on an electronic device. In addition, such a function can automatically activate in response to predetermined conditions (it is programmed to activate at a certain time, or in response to certain conditions). In some embodiments, a track function can be activated by another process remote from the subject (e.g., a server).

A display 306 can include all events displayed as icons on a visualization (e.g., graph) with a time scale 306-0. Each event icon can represent a particular activity and can be different for each activity. A display can also include health responses with the same event icons, at the same or partial time scale as that showing the events. In the embodiment shown, health responses can include glucose levels 306-1 and HR 306-2. In some embodiments, health responses can be a visualization (e.g., graph) showing health levels (e.g., glucose level, HR) over time, with activity icons overlaid thereon.

Event icons can be selectable by a subject. If a subject selects an event icon (Y from 308), details for the event can be displayed 310. As but some of many possible examples, event details can include text describing the event (food eaten, beverage consumed, physical activity performed, medication taken), the time and date of the event, and information particular to the event (e.g., calories, amount of water, calories burned, dose). If an event icon is not selected (N from 308), the information can continue to be displayed.

FIG. 3B shows an interactive display according to an embodiment. A display can be presented on a subject device in a method like that of FIG. 3A. The display shows, generally from top to bottom, a line graph with a time scale 340, above which can be displayed various event icons. Event icons can have different colors and/or symbols based on type (i.e., food, (physical) activity, medication, water). The time scale in the example shown is a 12-hour period, but such a time period can be increased or decreased.

The display can also show a visualization (e.g., graph) of BG levels 342 over a smaller time period. Overlaid on the curve representing BG levels can be icons (one shown as 342-0) for the various activities. The display can also show a graph of HR 346 over smaller time period. Overlaid on the curve representing HR levels can be icons (one shown as 344-0) for the various activities. In the embodiment shown, which events are displayed can be selectable by a subject. In the display of FIG. 3B as subject as selected “All” events, thus event icons for all types of events are shown. However, one particular type of event can be selected for display or in other embodiments, a subset of all possible even types can be selected for display.

### User Feed—Insight Cards

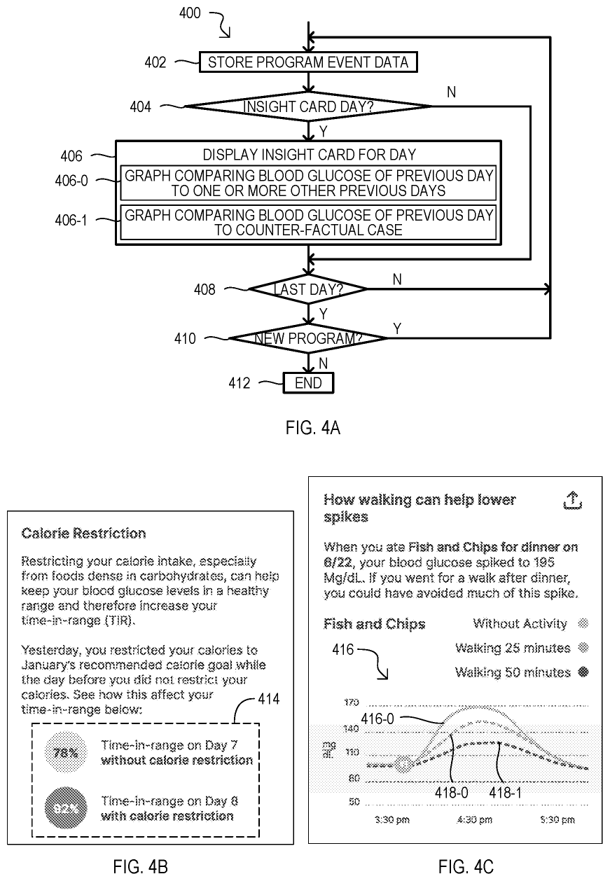

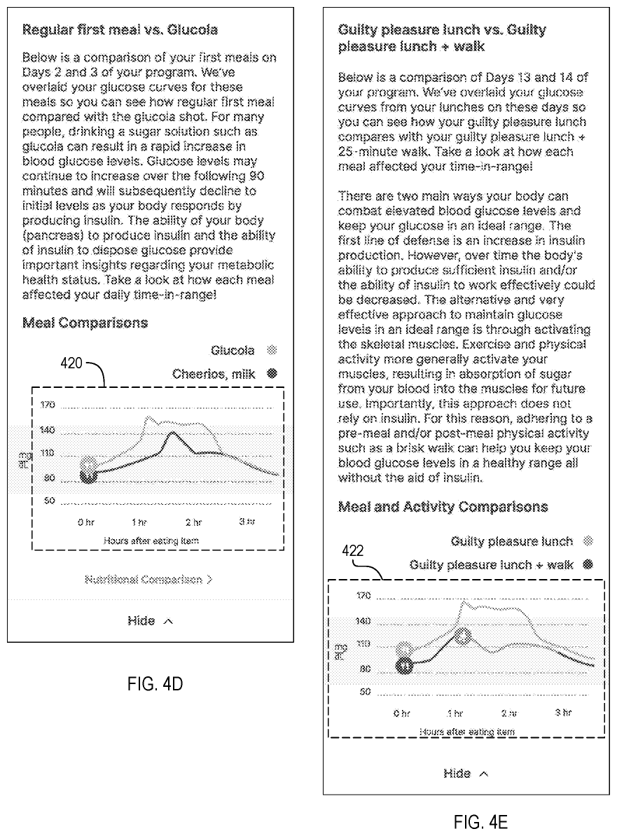

FIG. 4A is a flow diagram of a method 400 according to a further embodiment. A method 400 can monitor a subject's health related responses and present suggested activities, comparisons of subject activities to one another and/or to counterfactual activities. Presented suggestions can include graphical representation of the actual and/or expected health responses.

A method 400 can include storing program event data 402. Such an action can include storing event data according to any of the embodiments described herein. In addition, in the embodiment shown, events can follow a predetermined program, in which a subject performs certain activities on certain program days. In addition, according to the program, a subject will be presented with information to help track progress for one or more health goals (e.g., maintain BG levels in a range).

A method 400 can determine if a day is an insight card day 404. Such an action can include determining which day it is in the program. An insight day can be predetermined days within the program, including periodic occurrence. An insight day can also be event driven. If it is an “insight” day (Y from 404), a method 400 can activate a display (i.e., insight card) particular to that day 406. In the embodiment shown, such a display can include any of: a visualization (e.g., graph) comparing BG of a previous day of the program to one or more other days of the program 406-0 and/or a visualization (e.g., graph) comparing BG of a previous day to a counterfactual case 406-1. Various other displays can be presented as will be described below.

If it is not an insight card day (N from 404), or an insight card for a day has been generated, a method 408 can determine if a last day of the program has been reached 408. If a last day has been reached (Y from 408), a method 400 can determine if a subject will start a new program. If the subject will start a new program (Y from 410) or a last day of a program has not been reached (N from 408), a method 400 can return to 402. If a new program is not started (N from 410), a method 400 can end 412.

FIG. 4B shows an interactive display according to an embodiment. A display can be generated on a subject device in a method like that of FIG. 4A. The display shows, generally from top to bottom, the description of an activity (i.e., Calorie Restriction), followed by a comparison 414 of the health effect of the activity of BG levels (i.e., the amount of time BG was in a target range) for different days of the program (days 7 and 8 respectively). Such a comparison can include results when the activity is followed (with calorie restriction) and when the activity is not followed (without calorie restriction). In some embodiments, the health effects are selectable. For example, a user can select the 78% or 92% cases to see a more detailed representation of BG levels for the day (e.g., a graph).

FIG. 4C shows another interactive display according to an embodiment. Such a display can be generated in a method like that of FIG. 4A. The display shows, generally from top to bottom, the description of an activity (i.e., walking), followed by a comparison of the health effect of the activity as a counterfactual case (walking after a meal) with that of a program day (the meal without walking). In the embodiment shown, the BG response of the day is shown as a graph 416 over a time period showing an undesirable health response (e.g., increase in BG) 416-0. Overlaid on the graph is the projected BG response for one or more (in this case two) counterfactual cases 418-0/1 (e.g., with a 25 minute walk, with a 50 minute walk). In some embodiments, the various cases (actual and counterfactual) are selectable to view more detail.

FIG. 4D shows another interactive display for a method like that of FIG. 4A. The display shows, generally from top to bottom, the description of an activity (i.e., consumption of a clinical dose of a glucose solution, glucola) referenced to another day of a program. This is followed by a comparison between the activity (glucola on day 2), and a corresponding activity of another day (meal on day 3). In the embodiment shown, the comparison can include graphs 420 of BG levels the activities overlaid one another. In the embodiment shown, a nutritional comparison between the two activities can be selected for display.

FIG. 4E shows another interactive display according to an embodiment. Such a display can be generated in a method like that of FIG. 4A. The display shows, generally from top to bottom, the description of an activity of one day (i.e., guilty pleasure lunch) with that of another day (i.e., guilty pleasure lunch with a walk). This is followed by a graph 422 of BG responses for both days overlaid on one another. As shown, such a graphical representation can include icons for events (i.e., a walk). Such an event icon can be selectable for more information (e.g., day/time of walk, calories burned, walk geographical location etc.).

### Blood Glucose—Time-in-range, User Display, Suggestions for Mitigation

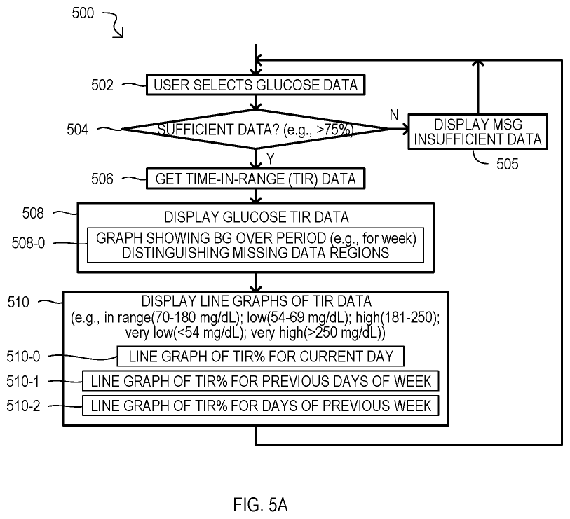

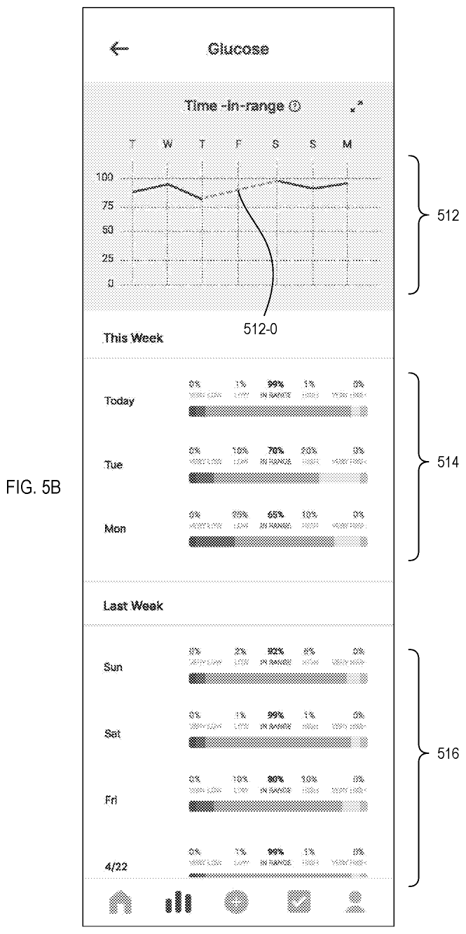

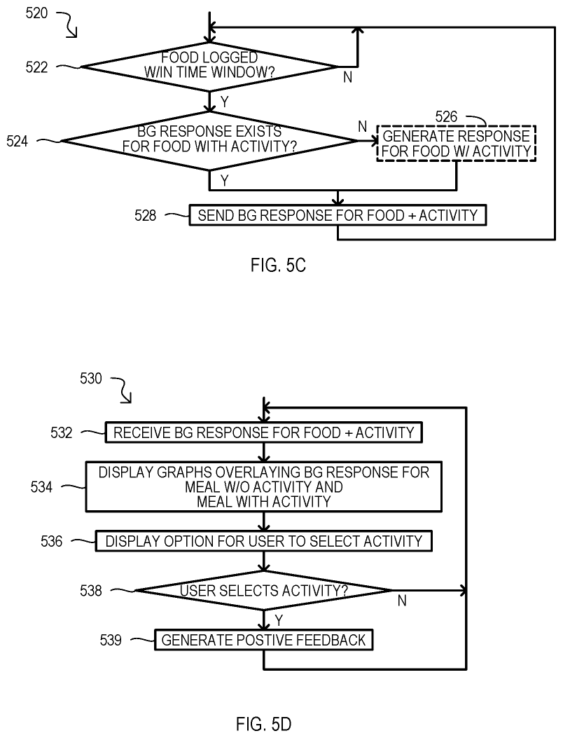

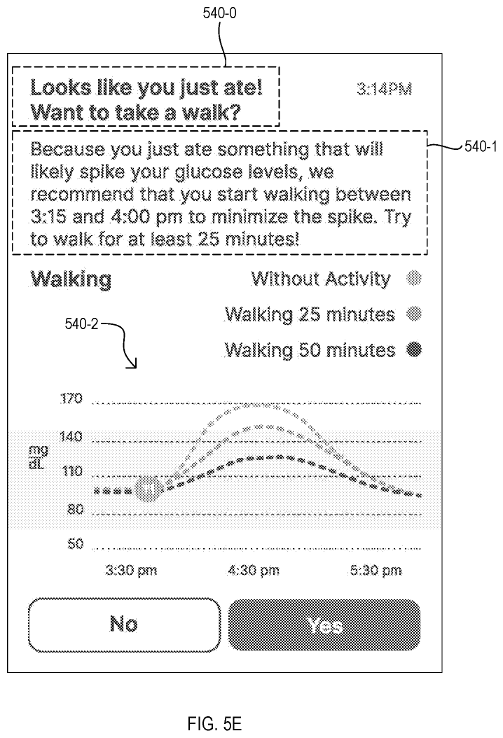

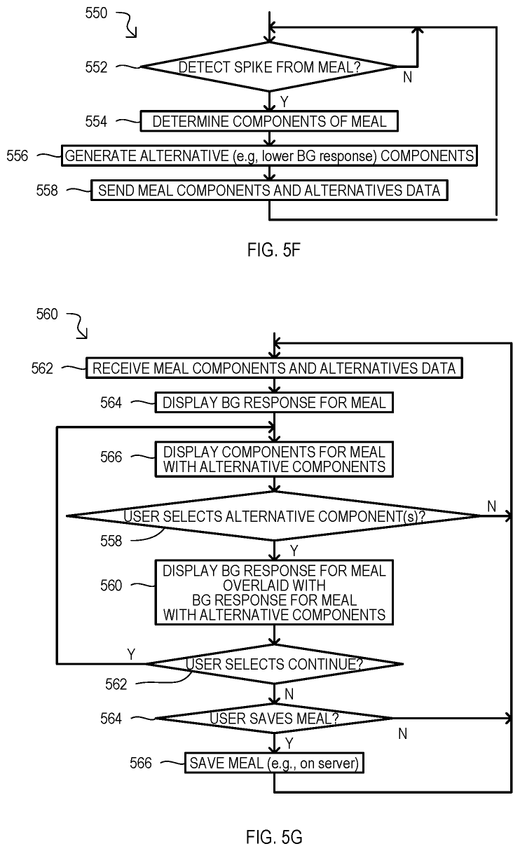

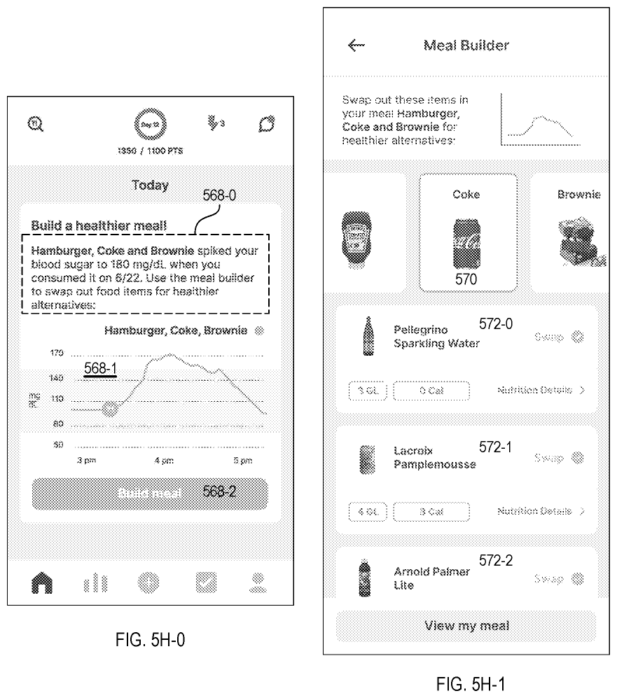

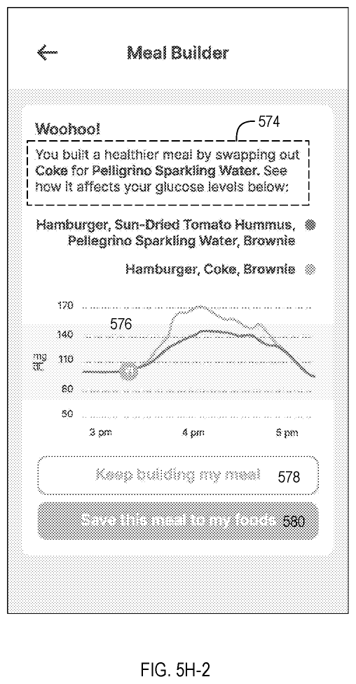

FIG. 5A is a flow diagram of a method 500 according to a further embodiment. A method 500 can monitor a subject's health related responses and determine if such responses are within a predetermined range. Data for the health-related response can presented for display on a user device showing present and historical levels. The embodiment of FIG. 5A shows a method for presenting time-in-range (TIR) data for blood glucose levels of a subject. A BG range for determining TIR can be a range particular to a subject based on a health goals or can be a range recommended by experts for all people, or for a subset of all people (e.g., a demographic). In some embodiments, a range for the TIR determination can be a target glucose response for a subject. A BG range for TIR determination can be a static value and/or dynamic value. A dynamic BG range can change in response to any of various circumstances, including but not limited to: a subject's change in health, time (e.g., time of day), subject activity, subject age, or subject medication, to name just a few.

A method 500 can be initiated by a subject selecting to view BG data 502. A determination can be made if sufficient BG data exists for a desired time period 504. In one embodiment, a time period can be a week, and the limits for sufficient data can be about 75% of the time period. However, alternate embodiments can include smaller or larger time ranges, as well as smaller or larger percentages. If sufficient BG data does not exist (N from 504), a message can be displayed on a subject device indicating so 505.

If sufficient data exists (Y from 504), TIR glucose data can be retrieved. Such an action can include a subject device retrieving data from a server, from a subject device, or a combination of both. Once TIR data has been retrieved, a method 500 can display glucose TIR data 508. While such a display can take any suitable form, in the embodiment shown, a graph can be displayed showing BG levels that were within a target range for a predetermined time period (e.g., a week) 508-0. In such a display, time periods in which BG data are missing can be distinguished from those time periods having BG data.

In addition, a method 500 can display line graphs of TIR data 510. In some embodiments, such a display can indicate multiple ranges, including “in range”, lower than desired range, higher than desired range, very low and very high. Such different ranges can be graphically distinguished from one another. In the embodiment shown, line graphs can indicate BG ranges as a percent of a current day 510-0, the previous days of the current week 510-1, and the previous days of the previous week 510-2.

FIG. 5B is a diagram of an interactive display showing BG TIR data according to an embodiment. A display can be generated on a subject device in a method like that of FIG. 5A. The display shows, generally from top to bottom, a graph 512 showing BG data for a week, including BG levels. Portions of the week in which BG data are missing are shown by a dashed line 512-0. The display also shows line graphs 514 for BG levels for days of the current week (This Week). The line graphs give percentages in which BG levels were within one of five different ranges (e.g., very low, low, in range, high, very high). Further, a bar is presented that is filled in by different colors corresponding to the different ranges. The display shows the same graphics for the previous week 516 (Last Week). In some embodiments, each of the days of the current week or previous week is selectable. When selected, more detailed information can be given for the BG levels for the day (e.g., a graph showing BG levels by hours, meals eaten, spiker events, etc.).

FIG. 5A shows a method 520 of generating a projected BG response according to an embodiment. A method 520 can be executed by a server in communication with a subject device, can be executed by the subject device, or can be executed by a combination thereof. A method 520 can include determining if food has been logged within a particular time window 522. Such an action can generate a difference between a food log time (e.g., time food was indicated as being eaten) to a current time. If such a time period is over a limit, the food logged can be considered outside of the window (N from 522). A time window can vary according to health goals, but in some embodiments can be a time period following a meal in which physical activity can mitigate a resulting rise in BG levels. In some embodiments, such a time period can be from about 30 min to 120 min.

If food has been logged within a window (Y from 522), a determination can be made as to whether BG data exists for the food eaten in conjunction with a physical activity 524. That is, has the subject's BG response for the same or similar meal along with, or followed by, physical activity been previously recorded. Such an action can include searching a subject's existing previous food logs, activity logs, HR logs, and corresponding BG data. In some embodiments, if there is no previous response (N from 524), a method 520 can generate such a response 526. Such an action can include applying the food and activity to a model which simulates a BG response. A model can take the form of any of those described herein, including preexisting models or models created by machine learning.

If a previous response exists (Y from 524) or a response is generated (526), data for the BG response and corresponding activity can be sent to a subject. Such an action can take any suitable form, including transmitting data from a server to a subject's electronic device and/or retrieving data on a subject device.

FIG. 5D shows a method 530 of presenting a proposed activity with corresponding beneficial health effects to a subject, in response to a subject's activity. In the embodiment shown, a physical activity is proposed in response to detecting a subject's consumption of food. A method 530 can be executed by a subject device, and can include receiving BG response data for a food and corresponding physical activity 532. Such an action can include receiving data from a server in response to entering food in a food log.

Once BG data for a food and physical activity has been retrieved, a visualization (e.g., graph) can be displayed the overlays a BG response for the food without the activity (e.g., meal only) along with a BG response for the food with the activity 534. An option for the user to select (e.g., verify) the physical activity can be presented 536. If the user does not select the activity (N from 538), a method 530 can return to 532.

If the user selects the activity (Y from 538), a method 530 can generate positive feedback 539. Positive feedback can take any suitable form, including but not limited to: a message, an option to post on a social medium, a reward (e.g., points that can be applied to acquire objects or services, monetary rewards, coupons, etc.).

FIG. 5E is a diagram of an interactive display showing a BG response of subject for a given food with and without a possible physical activity. A display can be generated on a subject device in a method like that of FIG. 5D. The display shows, generally from top to bottom, an acknowledgement 540-0 of the food eaten. A proposed activity, including optimal time is presented 540-1 for the subject (i.e., a walk between 3:15 and 4:00 pm). The display presents a graph 540-2 projecting BG levels of a subject if no physical activity is undertaken (Without Activity) as well as one or more (in this case two) physical activities are undertaken (Walking 25 minutes or 50 minutes). The various responses are distinguishable by different colors. The food event (meal) can also be shown as icon that is selectable for more detail. The display can also give the subject the option to select physical activity (No or Yes). If physical activity is selected, positive feedback can be generated as described herein.

FIG. 5F shows a method 550 of generating an alternate meal in response to detecting a meal consumed by a user. A method 550 can be executed by a server in communication with a subject device, executed by the subject device, or executed by a combination thereof. A method 550 can include detecting a meal that results in an undesirable BG response 552. In the embodiment shown, this can include detecting a spike in BG level. Such an action can include monitoring BG data from a subject for levels that exceed a limit, including absolute levels and/or rates of change. If such a meal is not detected (N from 552), method 550 can continue to monitor BG levels.

If such a meal is detected (Y from 552), method 550 can determine components of the meal 554. Such an action can include identifying meal components from a food log by accessing a database and/or using processes to derive meal components (which can include machine learning systems). Once components of the detected meal have been determined, a method 600 can generate alternative components 556. Such alternative components can be selected to have better health effect (e.g., lower BG response). In some embodiments, such an action can include a recommender system. The alternative meal components can then be sent to a subject 558. Such an action can take any suitable form, including transmitting data from a server to a subject's electronic device and/or retrieving data on a subject device.

FIG. 5G shows a method 560 of presenting a proposed alternative meal, in response to a subject's high BG meal. A method 560 can be executed by a subject's electronic device. In the embodiment shown, alternative meal components can be received 562. Such components can be generated according to a method like that of FIG. 5F, for example. Once alternative meal data has been received, a BG response for the meal consumed (i.e., the meal resulting in a high BG event) can be displayed 564. In addition, components for a meal with alternate components can be displayed 566.

If a subject selects a meal alternative (Y from 558), a BG response for the resulting alternative meal can be displayed, overlaid with that from the meal consumed 560. A subject can be given the option to explore other alternative meals/meal components 562. If a subject opts to continue (Y from 562), a method 560 can return to 566. If a subject opts not to continue (N from 562), a method 560 can give the subject the option to save the meal (with alternative components) 564. If a subject opts not to save a meal (N from 564), a method 560 can return to 562. If a subject opts to save a meal (Y from 564), the meal components can be saved 566. In some embodiments, this can be include transmitting a subject's selections to a server. The alternative meal can then be presented to a user in response to predetermined conditions.

FIGS. 5H-0 and 5H-1 are diagrams of an interactive display showing the presentation of alternate meal components to enable a subject to build an alternative meal to one that has been consumed. The alternative meal can have a lower BG response than the meal consumed. The display can be generated on a subject device in a method like that of FIG. 5G. Referring to FIG. 5H-0, the display shows, generally from top to bottom, a description of the meal consumed 568-0 and a line graph for BG levels resulting from the meal 568-1. A subject can be given the option to build an alternative meal (Build meal) 568-2.

Referring to FIG. 5H-1, a display is shown that can be presented if a subject opts to build a meal. The display shows components of the meal that was consumed (e.g., 570). Each meal component is selectable. When selected, one or more alternative meal component will be selected 572-0/1/2. Each alternative meal component can have a more beneficial effect on BG levels than the selected meal component.

Referring to FIG. 5H-2, a display is shown that can be presented if a subject constructs an alternative meal. The display shows components of the meal that have been substituted with alternative components 574. The display also shows a line graph 576 for BG levels resulting from the meal overlaid with projected BG levels resulting from the alternative meal created by the subject. A subject can be given the option to continue to explore alternative meal components (Keep building my meal) 578 or to save the created alternative meal (Save this meal to my foods) 580.

### Food Detection and Logging

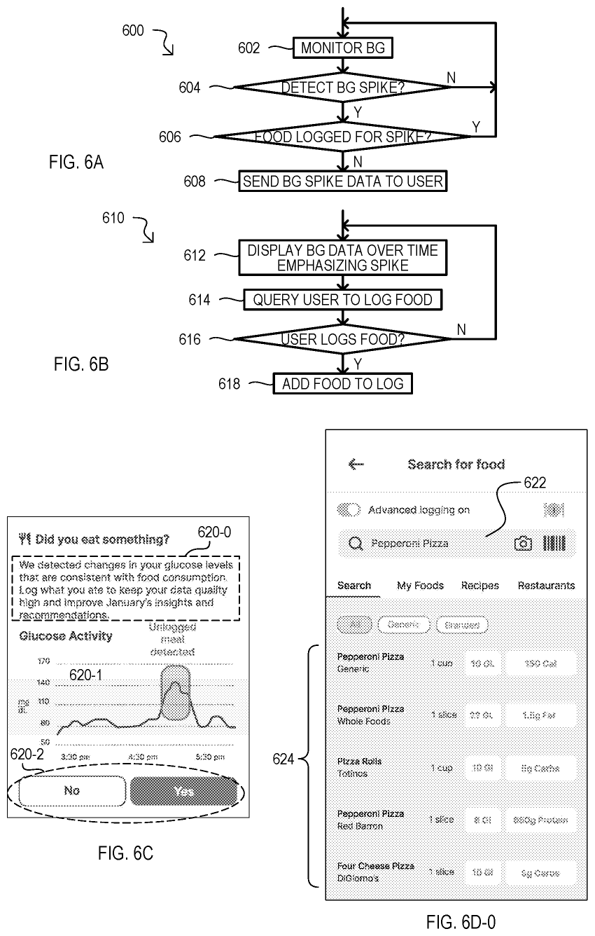

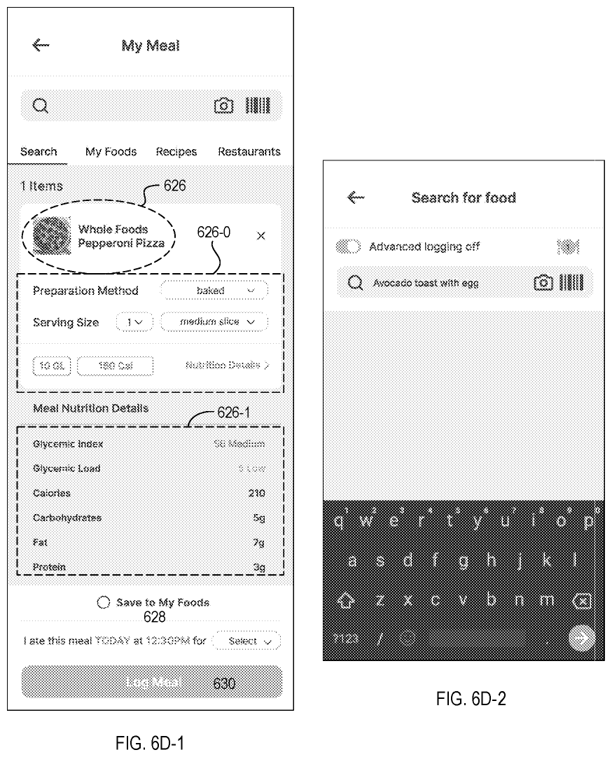

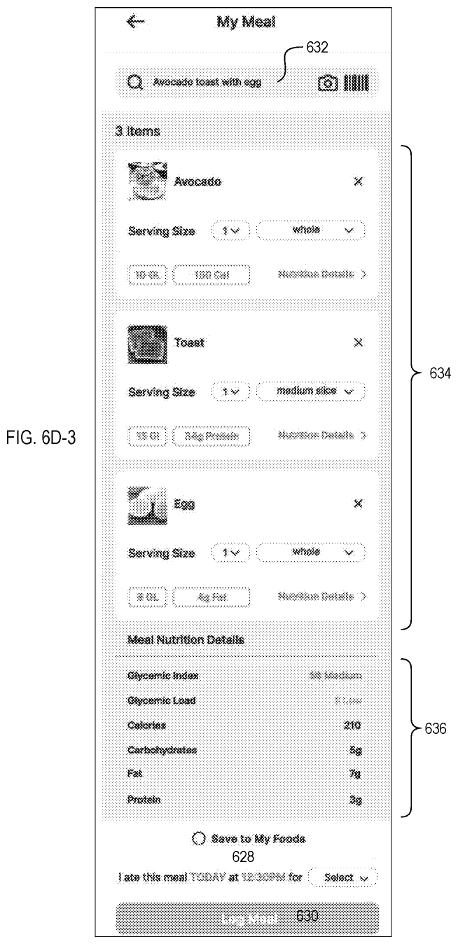

FIG. 6A is a flow diagram of a method 600 according to a further embodiment. A method 600 can monitor a subject's BG levels to determine if food is likely to have been eaten, and then prompt a subject to log food. A method 600 can be executed by a server in communication with a subject device, executed by the subject device, or executed by a combination thereof. A method 600 can include monitoring a glucose level of BG of subject 602. Such an action can take the form of any of those described herein. BG levels can be monitored for a predetermined increase (i.e., spike) 604. While there is no spike in levels (N from 604), monitoring can continue.

If a spike in BG levels is detected (Y from 604), a food log can be examined for a corresponding food 606 (i.e., a food that may have caused the spike). Such an action can include examining a food log for the presence of food within a predetermined time period of the BG spike. In some embodiments, such an action can be more complex and compare the BG response of the logged meal to an expected BG response for a same or similar meal. If there is sufficient deviation between the two BG responses, a food can be considered not to have been logged (i.e., erroneously logged). If food is considered to be logged (Y from 606), a method 600 can return to 602. If food is not considered to be logged (or logged erroneously) (N from 606), a method 600 can send data corresponding to the BG spike to a subject. Such an action can include a server sending data to a subject device, or a subject device retrieving such data.

In some embodiments, in response to food not being logged (N from 606), a method 600 can also derive a possible food corresponding to the BG data based on data from a subject. Data from a subject can include, but is not limited: previous meals eaten, user location, day, time of day, or like BG responses for which corresponding food data exists. A method 600 can transmit such derived food to the subject to enable easy selection for entry into a food log.

FIG. 6B shows a method 610 of notifying a subject of a possible missed food log event. A method 610 can be executed by an electronic device of a subject. A method 610 can include displaying BG data giving rise to the notice, emphasizing the spike in BG values 612. A subject can then be queried to log food for the BG spike event 614. If a user does not log any food (N from 616), a method 610 can return to 612. If a user logs food (Y from 616), a method 610 can enter such data into a food log of the user 618.

FIG. 6C is a diagram of an interactive display prompting a subject to enter food log data. The display can be generated on a subject device in a method like that of FIG. 6B. The display shows, generally from top to bottom, a message querying the subject 620-0 if food has been eaten and indicating that changes in blood glucose have been detected. The display also shows a graph 620-1 emphasizing BG response giving rise to the prompt. A subject can be given the option to confirm whether food was eaten but not yet logged (Yes/No) 620-2.

FIGS. 6D-1 to 6D-3 are diagrams of an interactive display showing food logging methods for a subject according to embodiments. The display can be generated on a subject device in a method like that of FIG. 6B. Referring to FIG. 6D-1, the display shows, generally from top to bottom, a text entry 622 for a subject to enter a food (filled with “Pepperoni Pizza” in the example shown). In response to entering text, a subject application can generate food matches 624. Such food matches can be based solely on the text, on the subject's food history, the subject's food preferences, the subject's geographical location (corresponding to the BG event), or combinations thereof. Searchable data can be restricted by the user to certain subsets, including foods noted by the subject (i.e., My Foods), recipes or restaurants. Food matches can be displayed with nutrition information (e.g., portions, glycemic response, calories). Food items are also selectable.

Referring to FIG. 6D-1, a display is shown that can be presented if a subject selects a food match, such as presented in FIG. 6D-0. The display shows, generally from top to bottom, the selected food 626, options regarding the food 626-0 (preparation method, serving size) and nutritional details for the food 626-1. The display can give the option to save the meal to a subject's meal list (Save to My Foods) 628 and/or to log the meal into the subject's food log (Log Meal) 630.

Referring to FIG. 6D-2, a display shows an alternative text entry to search for food. Such a text entry can be used to access a wider range of foods than that of FIG. 6D-1.

Referring to FIG. 6D-3, a display is shown that can be presented in response to a food search like that of FIG. 6D-2. The display shows, generally from top to bottom, the search terms used 632, search results (i.e., food matches) 634 with corresponding nutritional information 636 like that of FIG. 6D-0. As in the case of FIG. 6D-0, food matches can be selected to be saved 628 and/or logged 630.

### Tracking Fasting Periods

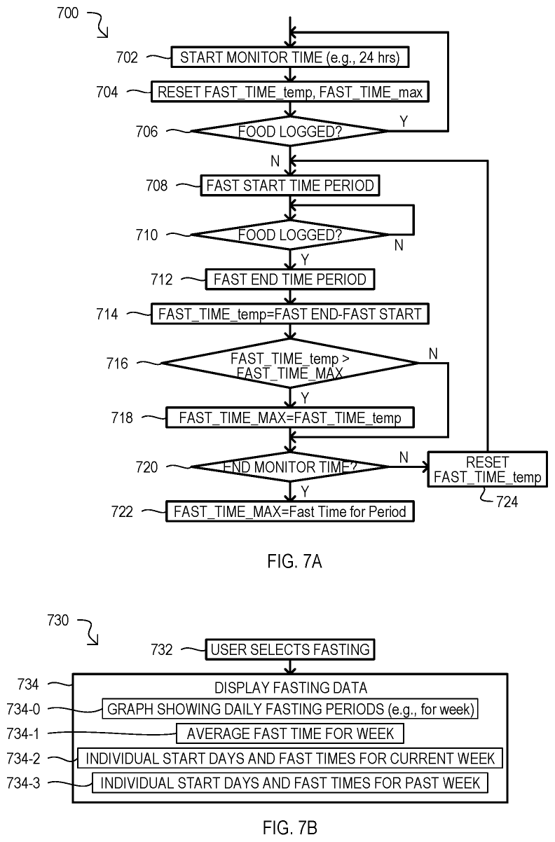

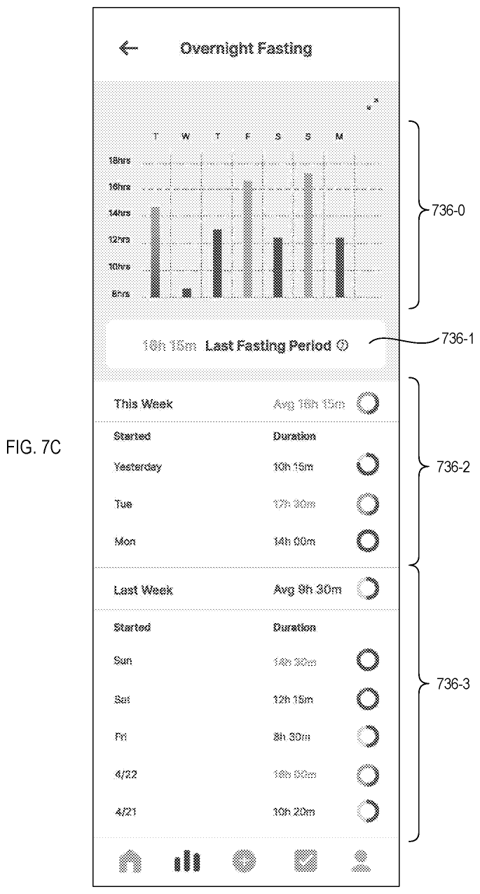

FIG. 7A is a flow diagram of a method 700 according to a further embodiment. A method 700 can monitor a subject's eating activity to identify fast periods and determine a longest fasting duration for a given time period. A method 700 can be executed by a server in communication with a subject device, executed by the subject device, or executed by a combination thereof. A method 700 can include starting a monitoring period 702. A monitoring period can be any suitable time, such as 24 hours as but one example. Temporary variables for a fast duration (FAST_TIME_temp, FAST_TIME_max) can be reset 704.

A method 700 can monitor a subject to determine whether food has been eaten 706. In the embodiment shown, this can include monitoring food logged (detecting food that food is not logged) by a subject. However, other embodiments can monitor other user data, including but not limited to BG levels. If it is determined that food has been eaten (Y from 706), a method 700 can return to 702.

If it has been determined that food has not been eaten (N from 706), a method can start a fast period 708. While in the fast period, a subject can be again monitored for food eaten 710. Once food is eaten (Y from 712), a fast time period can end 712. The duration of the fast period can be determined 714. If the fast duration exceeds an existing maximum fast duration (Y from 716), it can be set as the new maximum fast duration 718. If the fast duration does not exceed an existing maximum fast duration (N from 716) or a new maximum fast duration has been established 718, a method 700 can determine if the monitoring time period has ended 720. If the monitoring time period has not ended (N from 720), the method 700 can reset a current fast duration 724, and return to 708. If the monitoring time period has ended (Y from 720), the method 700 can set the current maximum fast duration to the fast duration of the time period 722.

FIG. 7B shows a method 730 of displaying fasting information for a subject according to an embodiment. A method 730 can be executed by an electronic device of a subject. A method 730 can include displaying fasting data 734, such as fasting data acquired by a method like that of FIG. 7A. Displayed fasting data 734 can include a graph showing daily fasting duration periods (i.e., durations) for a predetermined time period (e.g., a week) 734-0, an average fast time (i.e., duration) for a current week 734-1, individual start days and fast durations for a current week 734-2, and individual start days and fast durations for a past week 734-3.

FIG. 7C is a diagram of an interactive display for presenting fasting data. The display can be generated on a subject device in a method like that of FIG. 7B. The display shows, generally from top to bottom, a bar graph indicating fasting durations for each day of a current week 736-0 and a duration of a last fasting period 736-1. The display also shows the average duration of fasts for the current week 736-2, both in time values and a graphic, representing a proportion of a 24 hour day occupied by fasting. Start times and durations for the current week are displayed. The average duration of fasts for the previous week is also displayed, along with Start times and durations for the previous week 736-3.

### Calorie Restriction

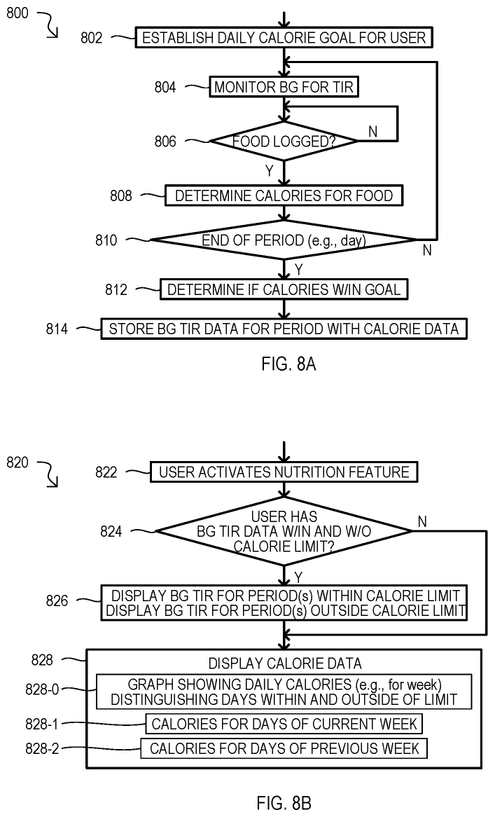

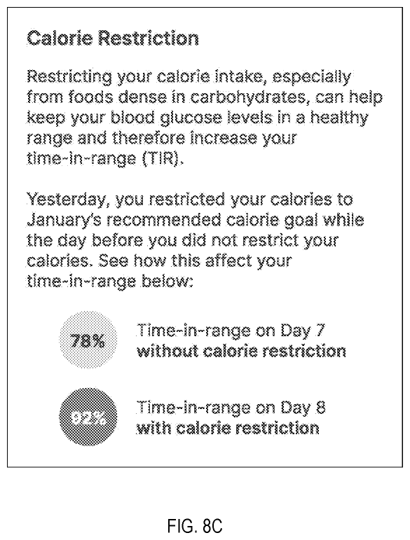

FIG. 8A is a flow diagram of a method 800 according to a further embodiment. A method 800 can monitor a subject's glucose levels and present data showing how glucose levels can be kept in range with calorie restriction. A method 800 can be executed by a server, a subject device, or a combination thereof. A method 800 can include establishing a calorie goal for a subject 802. A calorie goal can be a value particular to a subject based on a health goals or can be a value recommended by experts for all people, or for a subset of all people (e.g., demographic). A calorie goal can be for a predetermined time period (e.g., 24 hours). A subject can be monitored to determine a TIR of the glucose (BG) levels 804. A method can determine if food has been eaten 806. If food is not logged (N from 806), such monitoring can continue. If food is logged (Y from 806), the calories for the food can be determined 808.

A method 800 can determine if an analysis period has ended 810. If an analysis period has not ended (N from 810), a method 800 can return to 804. If an analysis period has ended (Y from 810), a method 800 can determine if calorie are within the established goal 812. BG TIR data and corresponding calorie data can be stored 814.

FIG. 8B shows a method 820 of displaying nutrition information for a subject according to an embodiment. A method 820 can be executed by an electronic device of a subject. A method 820 can be started by a subject activating a nutrition feature on an app 822. A determination can be made as to whether BG TIR data for a subject exists for time periods in which the subject was within a calorie limit (i.e., goal) and outside of a calorie limit 824. If such data exists (Y from 824), a method 820 can display BG TIR data distinguishing those time periods in which calories were within limits and not within limits. A method 820 can also include displaying calorie data 828. Displayed calorie data 828 can include a visualization (e.g., graph) showing daily calories. If there are time periods when calories were outside of limits 828-0, such time periods can be distinguished from in range time periods. In some embodiments, displayed calories can include calories for days of a current week 828-1 and for days of a previous week 828-2.

FIG. 8C is a diagram of an interactive display for presenting calorie data. The display can be generated on a subject device in a method like that of FIG. 7B. The display can show a message indicating that keeping calories restricted to a limit can result in better BG TIR. The display can also compare the BG TIR for a day in which calories were outside of limits (78%) compared to the BG TIR for a day in which calories were restricted to within the limit (92%).

### Metabolic Score

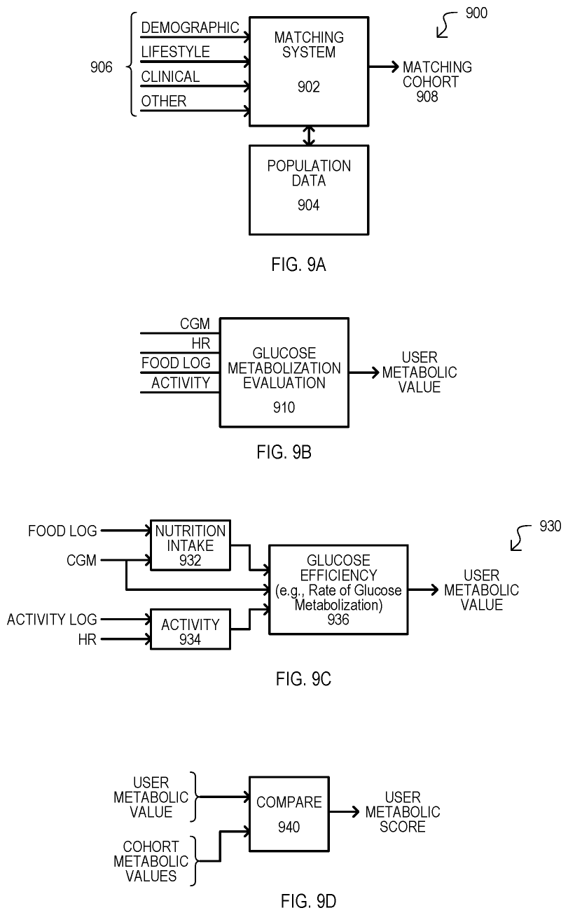

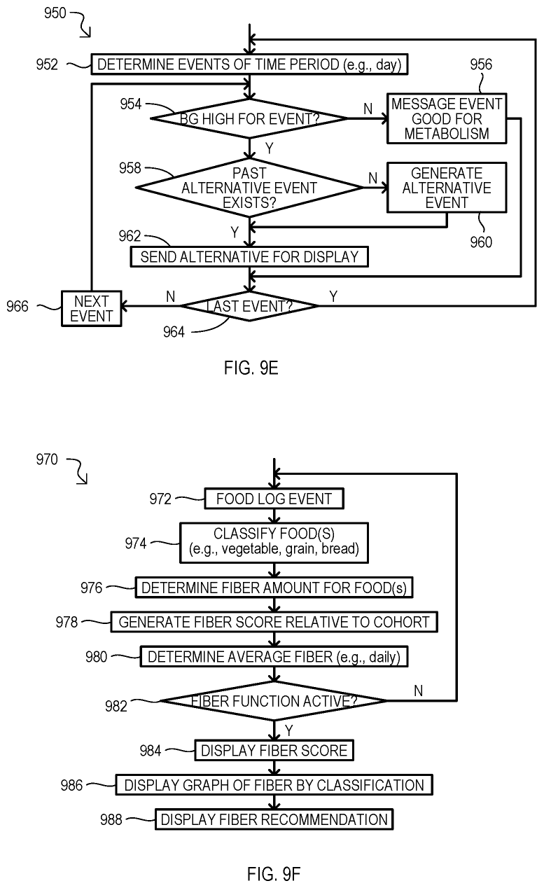

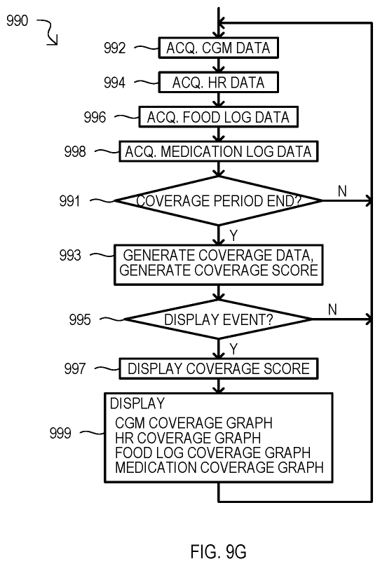

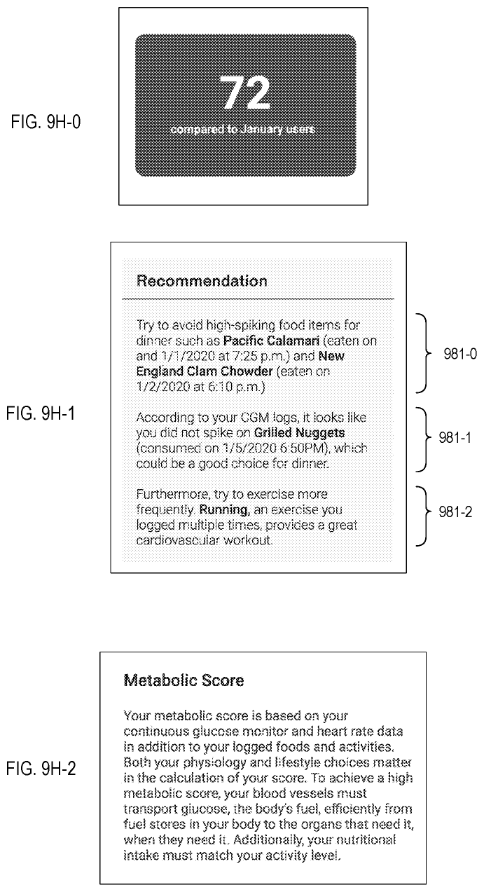

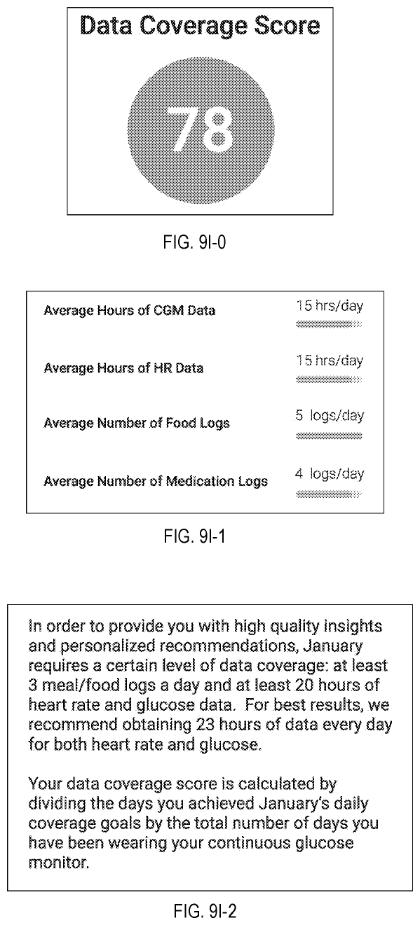

FIG. 9A is a block diagram of a system 900 according to an embodiment. A system 900 can identify cohorts to match a subject to a cohort. Such a cohort can be used as a comparison set to generate a metabolic score for a subject. A system 900 can include a matching system 902 and population data 904. In response to subject information 906, a matching system 902 can identify other subjects having metabolisms like that of the subject. Subject information 906 can include, but is not limited to, demographic data, lifestyle data, clinical data, and any other data related to a health related goal.

Based on subject information, a matching system 902 can access population and arrive at matches (persons who most closely match the subject's information), which can form a matching cohort 908.

FIG. 9B is a diagram showing the generation of a subject (i.e., user) metabolic value. A metabolic value can be generated by a glucose metabolization evaluation I200 based on user CGM data, HR data, food logs, and activity data. A glucose metabolization evaluation 910 can be according to an existing model, or can be a model derived with machine learning.

FIG. 9C is a block diagram showing a system and method 930 for the generation of a subject metabolic value according to a more detailed embodiment. A subject food log and CGM data can be used to arrive a nutrition intake value 932. A nutrition intake value 932 can correspond to an amount of metabolizable glucose for food taken in a given time period. A subject HR data and (optionally) activity log can be used to arrive at an activity value 934. An activity value 934 can be a reference value representing an expected glucose consumption rate. Using nutrition value 932 and activity value 934, and CGM data of the subject, a glucose burning efficiency value 936 can be determined. As but one example, such a value can correspond to a rate of glucose metabolization of the subject as compared to that of a reference value. This can be a subject metabolic value.

FIG. 9D is a block diagram showing the generation of a subject metabolic score according to an embodiment. A subject metabolic value (i.e., the value generated in FIGS. 9B and/or 9C), can be compared 940 to the same value generated for cohorts. Such a comparison can be metabolic score.

FIG. 9E is a diagram showing a method 950 according to an embodiment. Method 950 can evaluate a subject's actions and generate responses for improving a metabolic score for the subject. A method 950 can include determining subject events for a given time period 952. In some embodiments, a time period can be 24 hours, and events can include meals and activities and but two examples. An event can be examined to determine its effect on BG levels 954. If the event does not result in an increase in a target BG level or response (N from 954), a message can be generated or sent to indicate the event is good for metabolism 956. Such an action can include sending or generating a message on a subject device.

If the event does result in an increase in a target BG (Y from 954), a method 950 can determine if a past alternative for the event exists that has better effect on BG levels 958. Such an action can include examining a history of the subject, for a similar event and a lower resulting BG response. If no past alternative exists (N from 958), a method 950 can generate an alternative event know to have a better BG response 960. If a past alternative exists (Y from 958) or one is generated 960, the alternative can be sent for display on a subject's device 962.

Once an alternative is displayed 962 or a message 956 has been sent/displayed, a method 950 can determine if a last event has been reached 964. If a last event is not reached (N from 964), a next event can be analyzed 966 (then on to 954). If a last event is reached (Y from 964), a method 950 can return to 952.

FIG. 9F is a diagram showing a method 970 according to another embodiment. A method 970 can monitor food data and derive a fiber intake value and fiber source data for display on a subject device. A method 970 can include detecting a food log event 972. Such an action can include determining a subject has eaten food according to embodiments herein. If food is detected, the food can be classified into food type 974. A food type can include any suitable group, and in some embodiments can include vegetables, fruits, grain, bread, etc. An amount of fiber in the food can be determined 978. Such an action can include any suitable method, including accessing a food nutrition database.

A fiber score can be generated relative to a cohort 978. Such an action can include determining an amount of fiber consumed in a time period (e.g., a day) and comparing to that of a cohort. A cohort can be determined as shown in FIG. 9A. A subject's fiber intake can be rated against the fiber intake for the cohort to generate the fiber score. If a fiber function is not activated (N from 982), a method 970 can return to 972.

If a fiber function is activated (Y from 982), the fiber score can be displayed 984. In addition, a visualization (e.g., graph) can be displaced showing sources of fiber by classification 986. A fiber recommendation can also be displayed 988. Activating a fiber function can include selecting a function of an app running on a subject device.

FIG. 9G is a diagram showing a method 990 according to another embodiment. A method 990 can receive monitoring data from various reporting sources and generate a coverage value for display. A method 990 can include acquiring CGM data 992, acquiring HR data 994, acquiring food log data 996, and acquiring medication log data 998. All such actions can include receiving values that include accompanying time and date values. A method 990 can determine if a coverage monitoring period has ended 991. If such a period has not ended (N from 991), a method 990 can continue to acquire data (i.e., return to 992).

If a coverage period has ended (Y from 991), a method 990 can generate coverage data and a coverage score 993. Coverage data can represent coverage for various data sources over the coverage period. A coverage score can be a value representing data coverage for all sources with a single value. In some embodiments, such a value can be an average of the sources. However, in other embodiments such a value can include weighting some data sources over others.

A method 990 can determine if there is a display event 995. A display event 995 can include an app being activated to provide coverage data. Such an action can be in response to a subject selecting an app function or in response to an automatic display (e.g., daily report). Absent a display event (N from 995), a method 990 can continue to acquire and generate coverage data for a next coverage period (i.e., return to 992). In response to a display event (Y from 995), a method 990 can display a coverage score 997. In addition, coverage graphs for each data source can be displayed 999. A method 990 can then return to 992.

FIGS. 9H-0 to 9H-2 are diagrams of an interactive display showing metabolic scores and recommendations according to embodiments. Referring to FIG. 9H-0, a metabolic score is shown. The display can be generated by systems like those shown in FIGS. 9A to 9D. A metabolic score can show a subject's metabolism relative to a cohort.

Referring to FIG. 9H-1, a display shows metabolic recommendations that can be generated according to embodiments. The display can be generated by a method like that shown in FIG. 9E. FIG. 9H-1 shows, going generally from top to bottom, events that were determined to generate an adverse BG response (i.e., Pacific Calamari and New England Clam Chowder) 981-0; a past alternative event (Grilled Nuggets) suggested as an alternative to the adverse events 981-1; and a generated alternative event (Running) 981-2. Referring to FIG. 9H-2, a display provides a description of what a metabolic score represents and can accompany the score shown in FIG. 9H-0.

FIGS. 9I-0 to 9I-2 are diagrams of an interactive display showing coverage scores and values according to embodiments. Such scores and values can be generated by a method like that of FIG. 9G. Referring to FIG. 9I-0, a coverage score is shown. A coverage score was described with reference to FIG. 9G. FIG. 9I-1 shows coverage values for various data sources. Coverage values are represented by a visualization (e.g., graph) that is filled according to an amount of coverage. In the embodiment shown, coverage can be per day. FIG. 9I-2 provides a description of what a coverage score represents and can accompany the score shown in FIG. 9I-0.

### Lifestyle Score

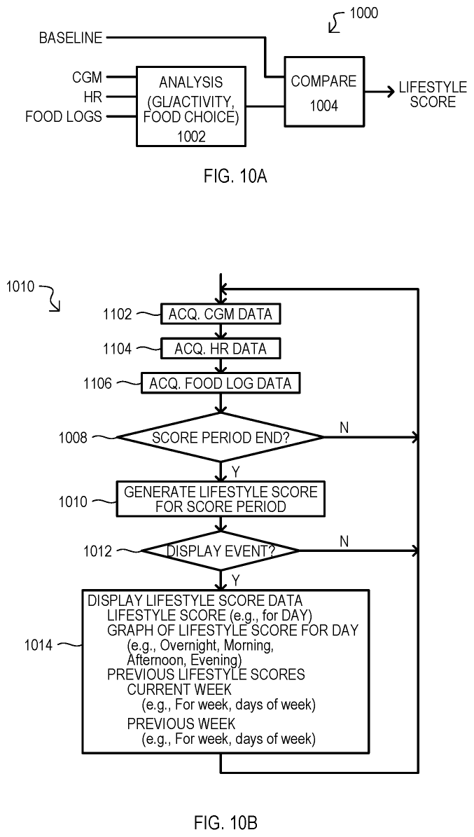

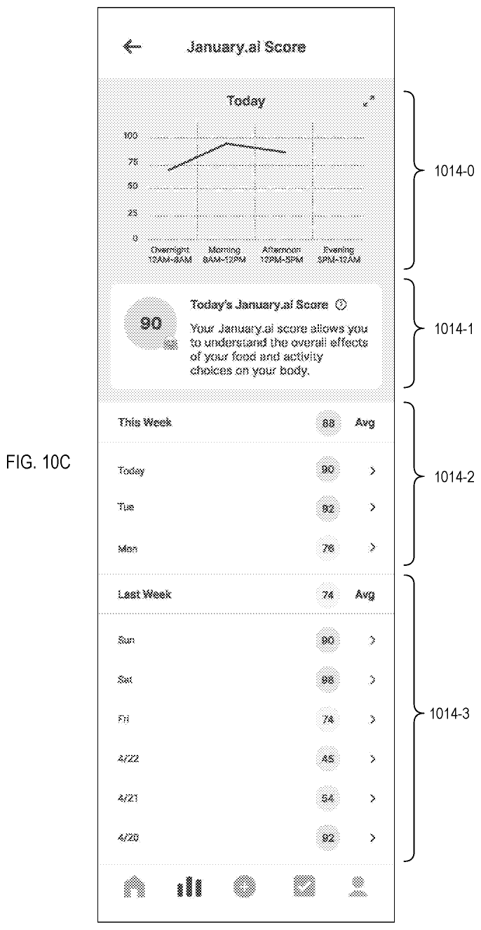

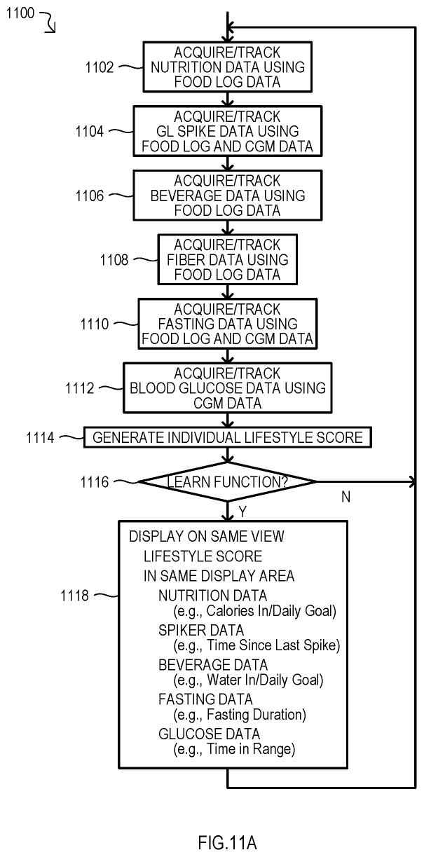

FIG. 10A is a diagram of a system 1000 according to an embodiment. A system 1000 can generate a lifestyle score for a subject based on choices of the subject. A system 1000 can include an analysis section 1002 and compare section 1004. An analysis section 1002 can receive CGM, HR and food log data for a subject. Such data can be analyzed to generate a value reflecting glucose levels (higher ratings for values that are in range, and do not spike), activity (higher rating for more activity), and food choices (higher rating for more nutritious food). A resulting value can be compared 1004 to a baseline value for the subject that can have been established previously. A baseline value can be generated by initial data (i.e., CGM, HR, food log), previous data, or any other suitable means (e.g., survey). Accordingly, as a subject makes choices that are better from a health perspective, a lifestyle score can improve.
FIG. 10B is a flow diagram of a method 1100 according to another embodiment. A method 1010 generate a lifestyle score data for presentation on a subject device. A method 1010 can include acquiring CGM data 1012, acquiring HR data 1014, and acquiring food log data 1016. A method 1010 can determine if a score period has ended 1018. If a score period has not ended (N from 1018), a method 1010 can continue to acquire data (i.e., return to 1012).

If a score period has ended (Y from 1018), a method 1010 can generate a lifestyle score for the score period 1010. Such an action can include a method like that shown in FIG. 10A. A method 1100 can include determining if there is a display event 1012. A display event 1012 can include an app being activated to provide a lifestyle score. Such an action can be in response to a subject selecting an app function or in response to an automatic display. Absent a display event (N from 1012), a method 1010 can continue to generate lifestyle score data for a next score period (i.e., return to 1012).

In response to a display event (Y from 1012), a method 1010 can display a lifestyle score data in various ways, including but not limited to: a lifestyle score for the current day, a visualization (e.g., graph) of a lifestyle score changes over the current day, an average lifestyle score for the week, lifestyle scores for other days of the current week, an average lifestyle score for the previous week, and lifestyle scores days of the previous week.

FIG. 10C is a diagram of an interactive display showing lifestyle score data according to an embodiment. A display can be generated on a subject device in a method like that of FIG. 10B. The display shows, generally from top to bottom, a graph 1014-0 showing a lifestyle score as it has change for days of a current week, a current lifestyle score (90) 1014-1, an average lifestyle for the current week as well as each day of the current week 1014-2, and the same for a previous week and each day of the previous week 1014-3. In some embodiments, each day of the current week or previous week is selectable. When selected, more detailed information can be given for the lifestyle score of that day (e.g., a graph showing how lifestyle scores changed over time, or lifestyle scores superimposed with event icons).

### Trending Data—Learn Pages

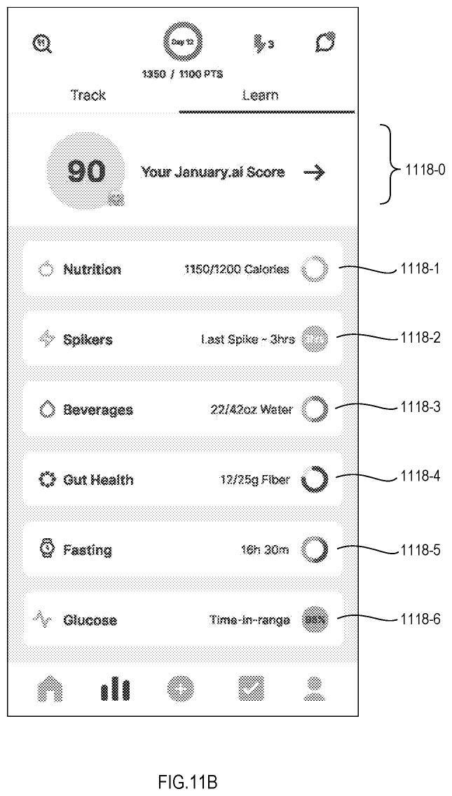

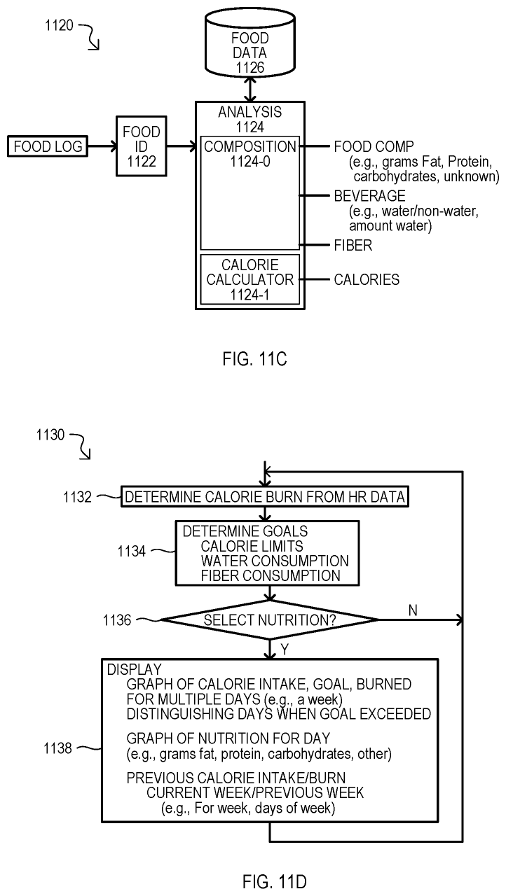

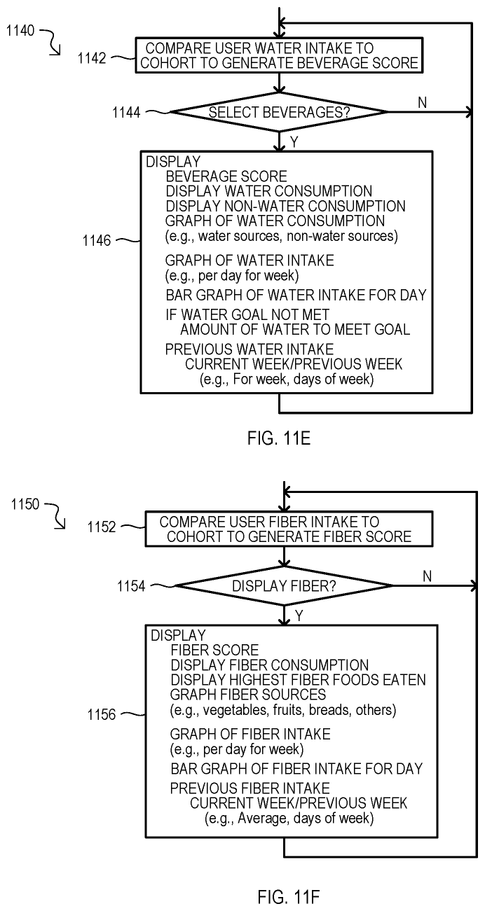

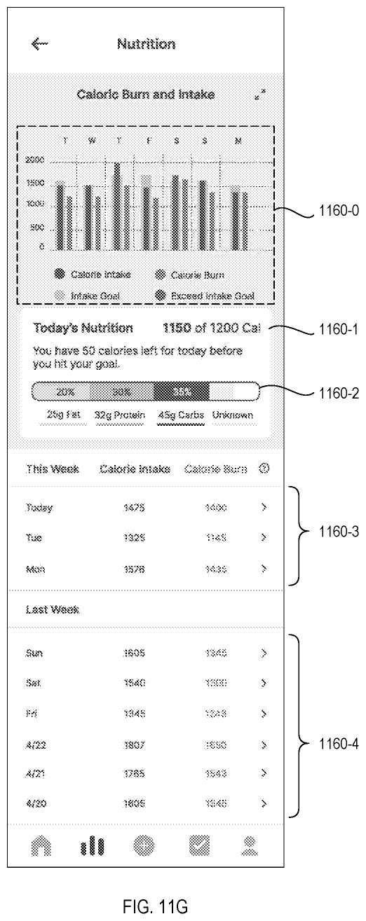

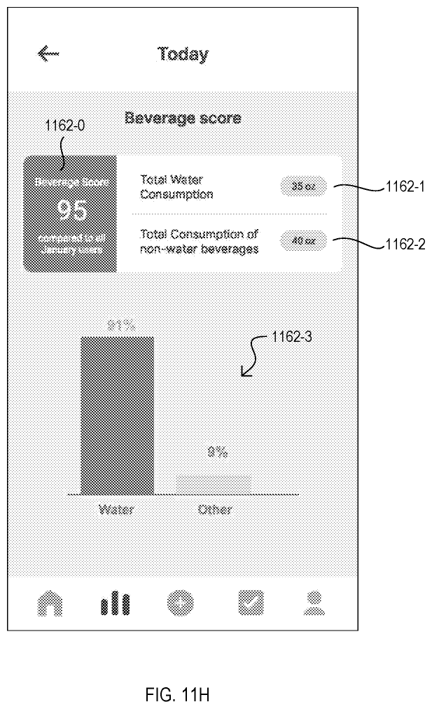

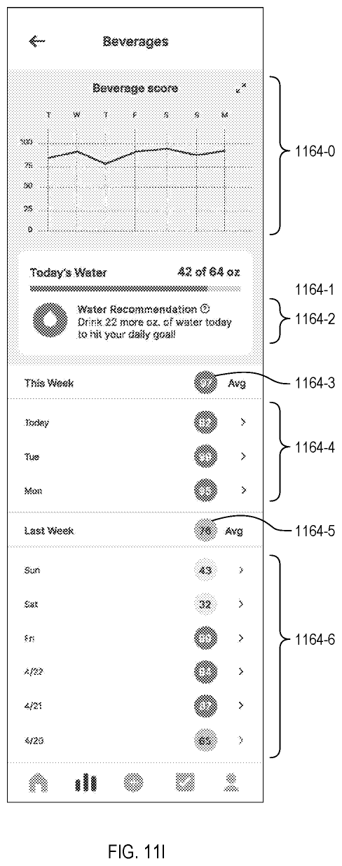

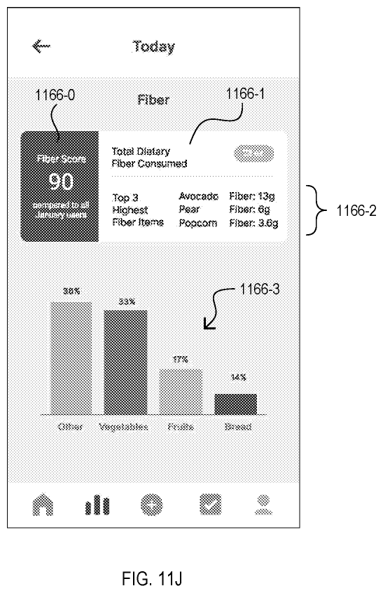

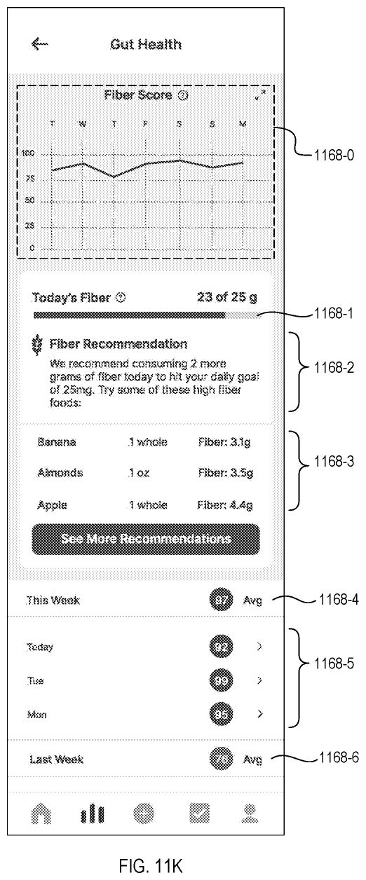

FIG. 11A is a flow diagram of a method 1100 according to an embodiment. A method 1100 can acquire and generate multiple values corresponding to a subject's health and lifestyle and present them in a single display. The values can be updated periodically to give a subject immediate information on trends and/or changes in such values. A method 1100 can include acquiring and tracking nutrition data using food log data 1102. In some embodiments, such an action can include those described for FIGS. 8A to 8C or 11D or equivalents. Data for glucose spiker events can be acquired and tracked with food log and CGM data 1104. In some embodiments, such an action can include those described for FIGS. 1A to 1E or equivalents. Data for beverages consumed can be tracked with food log data 1106. In some embodiments, such an action can include those described for FIG. 11D or an equivalent. Data for fiber consumed can be tracked with food log data 1108. In some embodiments, such an action can include those described for FIG. 9F or 11F or equivalents. Data for fasting can be acquired with food log data 1110. In some embodiments, such an action can include those described for FIGS. 7A to 7C or equivalents. Blood glucose data can be acquired with CGM data 1112. In some embodiments, such an action can include those described for FIGS. 5A and 5B or equivalents. An individual lifestyle score can be generated 1114. In some embodiments, such an action can include those described for FIGS. 10A to 10C or equivalents.

A method 1100 can determine if a learn function has been selected 1116. Selecting a learn function 1116 can include a subject selecting an app function, as but one example. Absent selection of the learn function (N from 1116), a method 1100 can continue to acquire/track data from the various sources and generate lifestyle values (return to 1102).

If the learn function is selected (Y from 1116), a method 1100 can include displaying on the same screen the various tracked and generated values, including but not limited to, the lifestyle score. In addition, in a same display area, can be displayed nutrition data, spiker data, beverage data, fasting data, and glucose data.

FIG. 11B is a diagram of an interactive display showing a learn function display according to an embodiment. A display can be generated on a subject device in a method like that of FIG. 11A. The display shows, generally from top to bottom, a lifestyle score 1118-0, a nutrition value 1118-1, spiker event data 1118-2, beverage data 1118-3, gut health (i.e., fiber) data 1118-4, fasting data 1118-5, and glucose data 1118-6. The various data displays are selectable, and when selected, can present more detailed information for the given value as described herein and equivalents.

FIG. 11C is a diagram of a food composition system 1120 according to an embodiment. A system 1120 can derive food composition data from data entered from a food log, or the like. A food composition system 1120 can include a food identification section 1122, which can generate a food identifying value having appropriate syntax for the system 1120. Food identifying information can undergo analysis 1124. Such analysis can take any suitable form, including database search and matching and/or machine learning systems. Analysis 1124 can include composition analysis 1124-0 and a calorie calculator 1124-1. In some embodiments, analysis 1124 can access a food database 1126 having nutritional information for foods.

Composition analysis 1124-0 can generate food composition information (e.g., grams of protein, fat, carbohydrates, and unknown in the event there is undetermined portions of a logged food), beverage information (e.g., which portions are or are not water), and fiber information. Calorie calculator 1124-1 can generate a calorie value.

FIG. 11D is a flow diagram of a method 1130 according to another embodiment. A method 1130 can generate a nutrition information for display on a subject device. A method 1130 can include determining calories burned from HR data 1132. A method 1130 can determine various health goals for a subject 1134 including calorie limitations, water consumption goals, and fiber consumption goals. A method K1130 can determine if a nutrition display (e.g., card) is selected 1136. Selecting a nutrition display 1136 can include a subject selecting an app function, as but one example. Absent selection of the nutrition display (N from 1136), a method 1130 can (return to K1132).

If the nutrition display is selected (Y from 1136), a method 1130 can include displaying a visualization (e.g., graph) of calorie intake versus calories burned for multiple days. Such a graph can distinguish days in which a calorie goal was met versus days the calorie was not met. A graph of nutrition for the day can be displayed. In some embodiments, such a graph can breakdown nutrition by grams (e.g., grams of protein, fat, carbohydrates or other). A display can also show calorie data for a previous week.

FIG. 11E is a flow diagram of a method 1140 according to another embodiment. A method 1140 can generate a beverage score reflecting water consumption for a subject. A method 1140 can include comparing water intake of a subject to water intake of a cohort to generate a beverage score 1142. A cohort can be as described herein, or equivalents. A method 1140 can include determining if a beverage display (e.g., card) is selected 1144. Selecting a beverage display 1144 can include a subject selecting an app function, as but one example. Absent selection of the beverage display (N from 1144), a method 1140 can return to 1142.

If the beverage display is selected (Y from 1144), a method 1140 can include displaying beverage information 1146. Beverage information can include the beverage score, water consumed, non-water consumed, a graph of water consumption, a graph of water intake, a bar graph of water intake for the day. If a water goal for day has not been met, the amount of water needed to meet the goal can be displayed. Previous water intake can also be displayed.

FIG. 11F is a flow diagram of a method 1150 according to another embodiment. A method 1150 can generate a fiber score reflecting fiber consumption for a subject. A method 1150 can include comparing fiber intake of a subject to fiber intake of a cohort to generate a fiber score 1152. A cohort can be as described herein, or equivalents. A method 1150 can include determining if a fiber display (e.g., card) is selected 1154. Selecting a fiber display 1154 can include a subject selecting an app function, as but one example. Absent selection of the fiber display (N from 1154), a method 1150 can return to 1152. If the fiber display is selected (Y from 1154), a method 1150 can include displaying fiber consumption information 1156. Fiber consumption information can include the fiber score, fiber consumed, a highest fiber food eaten, a graph of fiber sources, a graph of fiber intake, a bar graph of fiber intake for a day, and previous fiber intake.

FIG. 11G is a diagram of an interactive display for presenting calorie data. The display can be generated on a subject device in a method like that of FIG. 11D. The display shows, generally from top to bottom, a bar graph 1160-0 indicating a calorie intake, calories burned, and a calorie intake goal. The display also shows when a calorie intake exceeds a calorie goal. A display also shows how many calories have been consumed in the day with respect to a calorie intake goal (i.e., 1120 of 1200) 1160-1. The amount of calories remaining with respect to the goal can be displayed. Also displayed is a graph showing sources of calories 1160-2 as percentages of total calories. The display further shows, for single days, a calorie intake value and calorie burn value 1160-3. The days shown can include those days of the current week and those days of the previous week 1160-4. The days are selectable to view more detailed data about the calories consumed for that day.

FIG. 11H is a diagram of an interactive display for presenting beverage data. The display can be generated on a subject device in a method like that of FIG. 11E. The display shows, generally from top to bottom, a beverage score 1162-0 (which can be generated as described herein or an equivalent), a total amount of water consumed (i.e., 35 oz) 1162-1, and consumption of non-water beverages (i.e., 40 oz) 1162-2. The proportion of water to non-water of beverages consumed is also displayed in a bar graph 1162-3.

FIG. 11I is a diagram of an interactive display for presenting additional beverage data. The display can be generated on a subject device in a method like that of FIG. 11A or 11E. The display shows, generally from top to bottom, a graph of a beverage score for the week 1164-0, a line graph 1164-1 showing the amount of water consumed for the day, versus a water consumption goal, the amount of water needed 1164-2 to reach the goal is shown, a score for the present week 1164-3, scores for days of the week 1164-4, a score for the previous week 1164-5, and scores for the days of the previous week 1164-6. The days are selectable to view more detailed information about beverage consumption for the day.

FIG. 11J is a diagram of an interactive display for presenting fiber consumption data. The display can be generated on a subject device in a method like that of FIG. 11F. The display shows, generally from top to bottom, a fiber score 1166-0, which can be generated as described herein or an equivalent), a total amount of fiber consumed 1166-1, along with the top three highest fiber foods consumed 1166-2, identifying the food and its fiber content. A bar graph 1166-3 can be displayed showing proportion of fiber consumed with respect to particular source types (i.e., other, vegetables, fruits, bread).

FIG. 11K is a diagram of an interactive display for presenting additional fiber data. The display can be generated on a subject device in a method like that of FIG. 11A or 11F. The display shows, generally from top to bottom, a graph of a fiber score for the week 1168-0, the amount of fiber consumed for the day, versus a fiber consumption goal 1168-1, the amount of fiber needed to reach the daily goal 1168-2. The display further shows recommended high-fiber foods a subject may wish to try 1168-3. A subject has the option to see additional high-fiber food sources. Also displayed are fiber scores for a current week 1168-4, days of the current week 1168-5, and a previous week 1168-6. The days are selectable to view more detailed information about beverage consumption for the day.

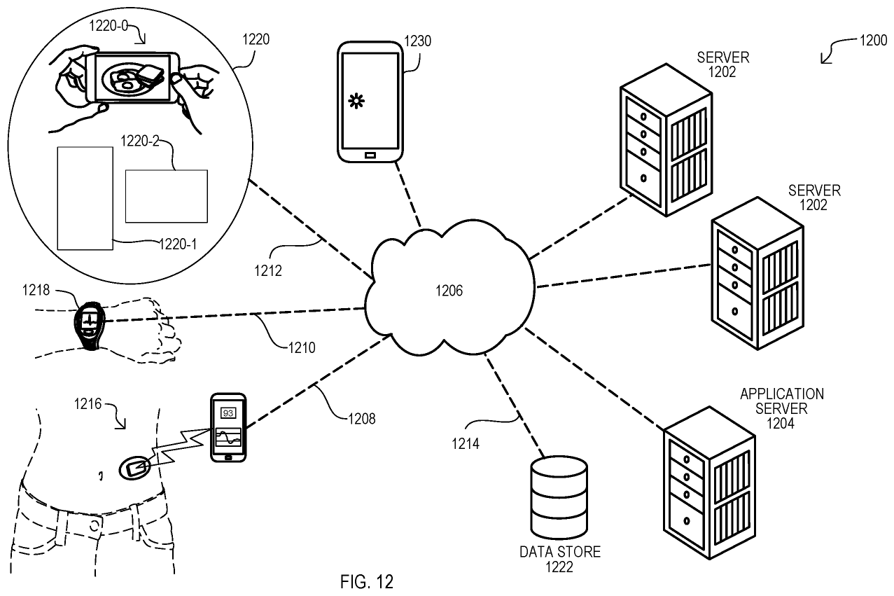

FIG. 12 shows a system 1200 according to an embodiment. A system 1200 can include one or more servers 1202, application servers 1204, data store 1222, multiple data sources (1208, 1210, 1212), and subject devices 1230. Data sources (1208, 1210, 1212), servers (1202, 1204) and subject devices 1230 can be in communication with one another, such as through a network 1206, which can include various interconnected networks, including the internet.

Servers (1202/1204) can execute various methods as described herein and equivalents. Such functions can acquire data from data sources (1216, 1218, 1220) as well as other data residing on data storage 1222.

An application server 1204 can interact with one or more applications running on a subject device 1230. In some embodiments, data from data sources (1216, 1218, 1220) can be acquired via one or more applications on a subject device (e.g., smart phone) and provided to application server 1204. Application server 1204 can communicate with subject device 1230 according to any suitable secure network protocol.

A data store 1222 can store data for system 1200. In some embodiments, data store 1222 can store data received from data sources (1216, 1218, 1220) (e.g., from subjects) as well as other data sets acquired by third parties. A data store 1222 can take any suitable form, including one or more network attached storage systems. In some embodiments, all or a portion of data store can be integrated with any of the servers (1202, 1204).

In some embodiments, data for data sources (1208, 1210, 1212) can be generated by sensors or can be logged data provided by subjects. In the example shown, data source 1208 can correspond to a first type sensor 1216, data source 1210 can correspond to a second type sensor 1218, and data source 1212 can correspond to logged data 1220 provided from a subject. Logged data 1220 can include data from any suitable source including text data as well as image data.

According to some embodiments, a first type sensor 1216 can be a “direct” data source, providing values for a biophysical subject response. A second type sensor 1218 and logged data 1220 can be “indirect” data sources. Such “indirect” data sources can be provided as inputs to system 1220. In some embodiments, a first type sensor 1216 can be a continuous glucose monitor (CGM), which can track a glucose level of a subject. A second type sensor 1218 can be heart rate monitor (HRM) which can track a subject's heart rate. Logged data 1220 can be subject nutrition data. Nutrition data 1220 can be acquired by an application on a subject device 1230. In some embodiments, image data can be captured, and such image data can be used to generate nutrition values. Image data can be images of text (e.g., labels 1220-1) which can be subject to optical character recognition to generate text, and such text can be applied to a server 1202 running an application which can identify a food. In addition or alternatively, image data can be images of actual food (e.g., 1220-0), or food packaging, and such image data can be applied to an application on a server for food identification. Further, logging can include capturing standardized labels (e.g., 1220-2) which can be subject to a database search or ML model to derive nutrition values.

A subject device 1230 can be any suitable device, including but not limited to, a smart phone, personal computer, wearable device, or tablet computing device. A subject device 1230 can include one or more applications that can communicate with application server 1222 to provide data to, and receive data from, biophysical models residing on ML servers 1202. Such application can take the form of, and/or include the functions of the various embodiments disclosed herein. In some embodiments, a subject device 1230 can be an intermediary for any of data sources (1208, 1210, 1212).

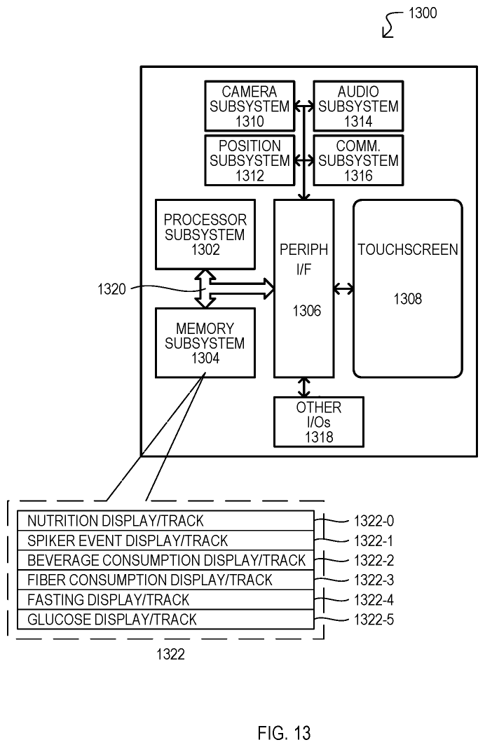

Various application functions and methods as described herein are executable on a subject device having a display screen. Such a subject device can include any suitable electronic device, and in some embodiments can include a portable electronic device, such as a smart phone or tablet device. FIG. 13 is a block diagram of a subject device 1300 according to embodiments.

A device 1300 can include a processor subsystem 1302, a memory subsystem 1304, a peripheral interface 1306, a display 1308, a camera subsystem 1310, a position subsystem 1312, an audio subsystem 1314, a communication subsystem 1316 and other input/outputs (I/Os) 1318. A processor subsystem 1302 can be connected to a memory subsystem 1304 by a bus system 1320.

A peripheral interface 1306 can serve to interconnect bus system 1320, various subsystems (1310, 1312, 1314, 1316), touchscreen 1308 and I/Os 1318.

A camera subsystem 1310 can include camera hardware and circuits for capturing images. A position subsystem 1312 can include sensors for determining a position, orientation and/or movement of device 1300. An audio subsystem 1314 can include hardware for generating and capturing sound. A communication subsystem 1316 can include circuits for communicating according to one or more wireless protocols, including but not limited those for a GSM network, an IEEE 802.11 wireless protocol (Wi-Fi) network, a Bluetooth network, etc.

A display 1308 can be touchscreen display and can facilitate the display of data and images for the various functions and applications described herein and equivalents. In addition, a touchscreen display can serve as an input for selecting various displays and functions and described herein and equivalents. Other I/Os 1318 can enable data inputs and outputs according to any suitable standard, including various serial data standards.

A processor subsystem 1302 can include one or more processor circuits for executing instructions stored in memory subsystem 1302.

A memory subsystem 1304 can include volatile memory, nonvolatile memory, and combinations thereof. In some embodiments, a memory subsystem 1304 can include an operating system and instructions for executing various subject device functions as described herein and equivalents. In some embodiments, a memory subsystem can include instruction for a health monitoring app as described herein and equivalents. In the embodiment shown, memory subsystem 1304 can include instructions 1322 for functions described herein, including but not limited to: displaying and tracking nutrition information 1322-0, displaying and tracking spiker events 1322-1, displaying and tracking beverage consumption 1322-2, displaying and tracking fiber consumption 1322-2, displaying and tracking fasting information 1322-4, and displaying and tracking glucose levels 1322-5.

It is understood that various blocks shown in the figures described herein can include any of various circuits configured to execute the indicated functions, including but not limited to servers systems that may or may not include customized hardware for accelerating operations, logic circuits, including custom logic circuits or programmable logic circuits. Such functions can also correspond to all or a portion of code executable by one or more processors that is stored on machine readable media. Data values as described herein can also be stored in machine readable media. Machine readable media can store code and/or data in a non-transitory form, in volatile and/or nonvolatile storage circuits.

It should be appreciated that in the foregoing description of exemplary embodiments of the invention, various features of the invention are sometimes grouped together in a single embodiment, figure, or description thereof for the purpose of streamlining the disclosure aiding in the understanding of one or more of the various inventive aspects. This method of disclosure, however, is not to be interpreted as reflecting an intention that the claimed invention requires more features than are expressly recited in each claim. Rather, as the following claims reflect, inventive aspects lie in less than all features of a single foregoing disclosed embodiment. Thus, the claims following the detailed description are hereby expressly incorporated into this detailed description, with each claim standing on its own as a separate embodiment of this invention.

It is also understood that the embodiments of the invention may be practiced in the absence of an element and/or step not specifically disclosed. That is, an inventive feature of the invention may be elimination of an element.

According to embodiments, blocks or actions that do not depend upon each other can be arranged or executed in parallel.

Accordingly, while the various aspects of the particular embodiments set forth herein have been described in detail, the present invention could be subject to various changes, substitutions, and alterations without departing from the spirit and scope of the invention.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

### Computer Systems

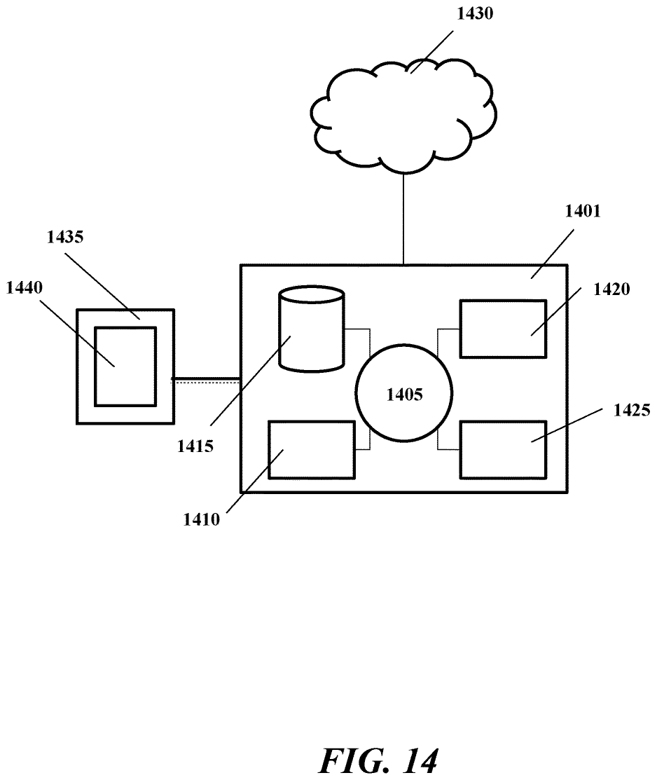

The present disclosure provides computer systems that are programmed to implement methods of the disclosure. FIG. 14 shows a computer system 1401 that is programmed or otherwise configured to perform signal processing, fuse sensor data, and perform machine learning operations. The computer system 1401 can regulate various aspects of contactless sleep monitoring of the present disclosure, such as, for example, performing machine learning tasks The computer system 1401 can be an electronic device of a user or a computer system that is remotely located with respect to the electronic device. The electronic device can be a mobile electronic device.

The computer system 1401 includes a central processing unit (CPU, also “processor” and “computer processor” herein) 1405, which can be a single core or multi core processor, or a plurality of processors for parallel processing. The computer system 1401 also includes memory or memory location 1410 (e.g., random-access memory, read-only memory, flash memory), electronic storage unit 1415 (e.g., hard disk), communication interface 1420 (e.g., network adapter) for communicating with one or more other systems, and peripheral devices 1425, such as cache, other memory, data storage and/or electronic display adapters. The memory 1410, storage unit 1415, interface 1420 and peripheral devices 1425 are in communication with the CPU 1405 through a communication bus (solid lines), such as a motherboard. The storage unit 1415 can be a data storage unit (or data repository) for storing data. The computer system 1401 can be operatively coupled to a computer network (“network”) 1430 with the aid of the communication interface 1420. The network 1430 can be the Internet, an internet and/or extranet, or an intranet and/or extranet that is in communication with the Internet. The network 1430 in some cases is a telecommunication and/or data network. The network 1430 can include one or more computer servers, which can enable distributed computing, such as cloud computing. The network 1430, in some cases with the aid of the computer system 1401, can implement a peer-to-peer network, which may enable devices coupled to the computer system 1401 to behave as a client or a server.

The CPU 1405 can execute a sequence of machine-readable instructions, which can be embodied in a program or software. The instructions may be stored in a memory location, such as the memory 1410. The instructions can be directed to the CPU 1405, which can subsequently program or otherwise configure the CPU 1405 to implement methods of the present disclosure. Examples of operations performed by the CPU 1405 can include fetch, decode, execute, and writeback.

The CPU 1405 can be part of a circuit, such as an integrated circuit. One or more other components of the system 1401 can be included in the circuit. In some cases, the circuit is an application specific integrated circuit (ASIC).

The storage unit 1415 can store files, such as drivers, libraries and saved programs. The storage unit 1415 can store user data, e.g., user preferences and user programs. The computer system 1401 in some cases can include one or more additional data storage units that are external to the computer system 1401, such as located on a remote server that is in communication with the computer system 1401 through an intranet or the Internet.

The computer system 1401 can communicate with one or more remote computer systems through the network 1430. For instance, the computer system 1401 can communicate with a remote computer system of a user (e.g., a mobile device). Examples of remote computer systems include personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants. The user can access the computer system 1401 via the network 1430.

Methods as described herein can be implemented by way of machine (e.g., computer processor) executable code stored on an electronic storage location of the computer system 1401, such as, for example, on the memory 1410 or electronic storage unit 1415. The machine executable or machine readable code can be provided in the form of software. During use, the code can be executed by the processor 1405. In some cases, the code can be retrieved from the storage unit 1415 and stored on the memory 1410 for ready access by the processor 1405. In some situations, the electronic storage unit 1415 can be precluded, and machine-executable instructions are stored on memory 1410.

The code can be pre-compiled and configured for use with a machine having a processer adapted to execute the code, or can be compiled during runtime. The code can be supplied in a programming language that can be selected to enable the code to execute in a pre-compiled or as-compiled fashion.

Aspects of the systems and methods provided herein, such as the computer system 1401, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 1401 can include or be in communication with an electronic display 1435 that comprises a user interface (UI) 1440 for providing, for example, a method for configuring machine learning algorithms. Examples of UI's include, without limitation, a graphical user interface (GUI) and web-based user interface.

Methods and systems of the present disclosure can be implemented by way of one or more algorithms. An algorithm can be implemented by way of software upon execution by the central processing unit 1405. The algorithm can, for example, create a latent representation of sensor data.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

What is claimed is:

1. A personalized glucose monitoring device for a test subject, comprising:
a heart rate monitor configured to obtain time-series heart rate data of the test subject over a period of time;
a continuous glucose monitor configured to obtain time-series blood glucose levels of the test subject over the period of time;
computer memory configured to store a set of training data comprising food source data for a set of training subjects and glucose levels responsive to the food source data; and
one or more computer processors operatively coupled to the computer memory, wherein the one or more computer processors are individually or collectively programmed to:
(i) train a starting glucose prediction model with the set of training data to predict blood glucose levels in response at least in part to food source data, the starting glucose regulation model comprising population-based insulin resistance parameters, wherein the starting glucose regulation model comprises an artificial neural network configured to analyze time-series input data, wherein the artificial neural network comprises (i) an input layer configured to receive a sequence of the time-series input data and (ii) and one or more hidden layers that are trained to maintain a state across time increments and capture dependencies in the sequence of the time-series input data;
(ii) obtain personalized glycemic response data of the test subject, wherein obtaining the personalized glycemic response data comprises obtaining the time-series heart rate data of the test subject from the heart rate monitor, and obtaining the time-series blood glucose levels of the test subject from the continuous glucose monitor;
(iii) update the starting glucose regulation model with the personalized glycemic response data of the test subject, wherein the updating comprises adjusting the population-based insulin resistance parameters, until convergence is achieved between a predicted glycemic response of the test subject and an actual glycemic response of the test subject, thereby producing a personalized glucose regulation model for the test subject;
(iv) generate a predicted blood glucose level for the test subject, using the personalized glucose regulation model for the test subject;
(v) automatically generate a message containing the predicted blood glucose level for the test subject whenever an updated predicted blood glucose level has been generated; and
(vi) transmit the message to all users of the personalized glucose monitoring device over a computer network in real time, so that each user has real-time access to up-to-date predicted blood glucose information for the test subject.

2. The personalized glucose monitoring device of claim 1, wherein the starting glucose regulation model comprises a differential equation model or a set of coupled equations.

3. The personalized glucose monitoring device of claim 1, wherein the one or more computer processors are individually or collectively programmed to further apply the personalized glucose regulation model for the test subject to at least personal food source data of the test subject to generate the predicted blood glucose level for the test subject.

4. The personalized glucose monitoring device of claim 3, wherein the one or more computer processors are individually or collectively programmed to further generate the predicted blood glucose level for the test subject in real time.

5. The personalized glucose monitoring device of claim 1, wherein the artificial neural network comprises a recurrent neural network (RNN).

6. The personalized glucose monitoring device of claim 1, wherein the artificial neural network is trained with data of a pre-determined population.

7. The personalized glucose monitoring device of claim 1, wherein (iii) further comprises classifying the test subject into a demographic equivalent group based at least in part on characteristic data of the test subject.

8. The personalized glucose monitoring device of claim 2, wherein the differential equation model comprises a food source function, a glucose production function, or a glucose uptake function.

9. The personalized glucose monitoring device of claim 8, wherein the one or more computer processors are individually or collectively programmed to further train the food source function using training data comprising glycemic responses of a population to pre-determined foods or glycemic responses calculated from data for pre-determined foods.

10. The personalized glucose monitoring device of claim 5, wherein the RNN comprises a long short-term memory (LSTM) RNN.

11. The personalized glucose monitoring device of claim 10, wherein the LSTM RNN comprises a set of LSTM units, wherein each of the set of LSTM units comprises a cell, an input gate, an output gate, and a forget gate.

12. The personalized glucose monitoring device of claim 1, wherein the one or more computer processors are individually or collectively programmed to further generate a recommended action for the test subject, based at least in part on the predicted blood glucose level for the test subject.

13. The personalized glucose monitoring device of claim 12, wherein the recommended action comprises a medical recommendation, a diet recommendation, a physical activity recommendation, a sleep recommendation, a hydration recommendation, or a stress release recommendation.

14. The personalized glucose monitoring device of claim 1, wherein the one or more computer processors are individually or collectively programmed to further:
(vii) determine whether the predicted blood glucose level for the test subject has a deviation outside of response limits; and
(viii) responsive to the determining in (vii), generate a notification indicating whether the predicted blood glucose level for the test subject has the deviation outside of the response limits.

15. The personalized glucose monitoring device of claim 14, wherein the notification is sent to the test subject.

16. The personalized glucose monitoring device of claim 14, wherein the notification is sent to a party different from the test subject.

17. A method for personalized glucose monitoring of a subject, comprising:
(a) using a heart rate monitor to obtain time-series heart rate data of the subject over a period of time;
(b) using a continuous glucose monitor to obtain time-series blood glucose levels of the subject over the period of time;
(c) obtain a personalized glucose regulation model for the subject, wherein the personalized glucose regulation model is trained at least in part by:
(1) obtaining a starting glucose prediction model configured to predict blood glucose levels in response at least in part to food source data, the starting glucose regulation model comprising population-based insulin resistance parameters, wherein the starting glucose regulation model is configured to analyze time-series input data;
(2) obtaining personalized glycemic response data of the subject, wherein obtaining the personalized glycemic response data comprises obtaining the time-series heart rate data of the subject from the heart rate monitor, and obtaining the time-series blood glucose levels of the subject from the continuous glucose monitor; and
(3) updating the starting glucose regulation model with the personalized glycemic response data of the subject, wherein the updating comprises adjusting the population-based insulin resistance parameters, until convergence is achieved between a predicted glycemic response of the subject and an actual glycemic response of the subject;
(d) generate a predicted blood glucose level for the subject in real time, using the personalized glucose regulation model for the subject;
(e) automatically generate a message containing the predicted blood glucose level for the subject whenever an updated predicted blood glucose level has been generated; and
(f) transmit the message to a user set of the personalized glucose monitoring device over a computer network in real time, so that each user of the user set has real-time access to up-to-date predicted blood glucose information for the subject.

18. A method, comprising:
obtaining a blood glucose level of a subject;
when the blood glucose level of the subject deviates from a target response,
determining a food consumed by the subject that corresponds to the deviated blood glucose level, and
generating at least one alternative action for the subject, wherein the at least one alternative action generates a blood glucose response in the subject with a reduced deviation from the target response as compared to a second glucose response generated by the subject consuming the food in absence of the at least one alternative action; and
electronically displaying at least the at least one alternative action.
